(19) United States
(12) Patent Application Publication
(10) Pub. No.: US 2003/0064919 A1
(43) Pub. Date: Apr. 3, 2003

(54) Novel polypeptides and polynucleotides encoding same

(75) Inventors: Sudhirdas K. Prayaga, O'Fallon, MO (US); Richard A. Shimkets, West Haven, CT (US)

Correspondence Address: Ivor R. Elrifi, Ph.D., Mintz, Levin, Cohn, Ferris, Glovsky & Popeo, P.C., One Financial Center, Boston, MA, 02111, US

(73) Assignee: CURAGEN CORPORATION

(21) Appl. No.: 09/732,436

(22) Filed: Dec. 7, 2000

Related U.S. Application Data
(60) Provisional application No. 60/169,887, filed on Dec. 9, 1999.
(60) Provisional application No. 60/170,230, filed on Dec. 10, 1999.

(51) Int. Cl. A61K 48/00; A61K 38/17; C07H 21/04; C12N 9/00; C12P 21/02; C12N 5/06; C07K 14/435

(52) U.S. Cl. 514/12; 514/44; 435/69.1; 435/325; 435/320.1; 435/183; 530/350; 536/23.2

(57) ABSTRACT

The invention provides polypeptides, designated herein as NOV polypeptides, as well as polynucleotides encoding NOV polypeptides, and antibodies that immunospecifically-bind to NOV polypeptide or polynucleotide, or derivatives, variants, mutants, or fragments thereof. The invention additionally provides methods in which the NOV polypeptide, polynucleotide, and antibody are used in the detection, prevention, and treatment of a broad range of pathological states.

### RELATED APPLICATIONS

This application claims priority to U.S. Ser. No. 60/169,887, filed Dec. 9, 1999 and U.S. Ser. No. 60/170,230, filed Dec. 10, 1999. The contents of these applications are incorporated herein by reference in their entireties.

### BACKGROUND OF THE INVENTION

The invention generally relates to nucleic acids and polypeptides encoded therefrom. More specifically, the invention relates to nucleic acids encoding membrane bound and secreted polypeptides, as well as vectors, host cells, antibodies, and recombinant methods for producing these nucleic acids and polypeptides.

### SUMMARY OF THE INVENTION

The invention is based in part upon the discovery of three novel nucleic acid sequences encoding membrane bound and secreted polypeptides. Nucleic acids encoding these polypeptides and derivatives and fragments thereof, will hereinafter be collectively designated as “NOV.”

In one aspect, the invention provides an isolated NOV1 nucleic acid molecule encoding a NOV1 polypeptide that has identity to the polypeptide sequence for the human immunoregulatory protein interferon omega-1. The invention also provides an isolated NOV2 nucleic acid molecule encoding a NOV2 polypeptide that is a novel transmembrane polypeptide.

In some embodiments, the NOV nucleic acid molecule can hybridize under stringent conditions to a nucleic acid sequence complementary to a nucleic acid molecule that includes a protein-coding sequence of the nucleic acid sequence. Also included in the invention is an oligonucleotide, e.g., an oligonucleotide which includes at least 6 contiguous nucleotides of a NOV nucleic acid (e.g., SEQ ID NO:1, 3, or 5) or a complement of said oligonucleotide.

Also included in the invention are substantially purified NOV polypeptides (SEQ ID NO:2, 4, or 6. The invention also features antibodies that immunoselectively-bind to NOV polypeptides.

In another aspect, the invention includes pharmaceutical compositions which include therapeutically- or prophylactically-effective amounts of a therapeutic and a pharmaceutically-acceptable carrier. The therapeutic can be, e.g., a NOV nucleic acid, a NOV polypeptide, or an antibody specific for a NOV polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOV nucleic acid, under conditions allowing for expression of the NOV polypeptide encoded by the DNA. If desired, the NOV polypeptide can then be recovered.

In another aspect, the invention includes a method of detecting the presence of a NOV polypeptide in a sample. In the method, a sample is contacted with a compound that selectively binds to the polypeptide under conditions allowing for formation of a complex between the polypeptide and the compound. The complex is detected, if present, thereby identifying the NOV polypeptide within the sample.

Also included in the invention is a method of detecting the presence of a NOV nucleic acid molecule in a sample by contacting the sample with a NOV nucleic acid probe or primer, and detecting whether the nucleic acid probe or primer bound to a NOV nucleic acid molecule in the sample.

In a further aspect, the invention provides a method for modulating the activity of a NOV polypeptide by contacting a cell sample that includes the NOV polypeptide with a compound that binds to the NOV polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes outlined in the preferred embodiment below. The therapeutic can be, e.g., a NOV nucleic acid, a NOV polypeptide, or a NOV-specific antibody, or biologically-active derivatives or fragments thereof.

In the preferred embodiments, the invention further includes methods for screening for a modulator of disorders or syndromes including, e.g., those involving development, differentiation, and activation of thymic immune cells; in pathologies related to spermatogenesis and male infertility; diagnosis of several human neoplasias; in diseases or pathologies of cells in blood circulation such as red blood cells and platelets; various immunological disorders and/or pathologies; autoimmune and inflammatory diseases; cardiovascular diseases; metabolic diseases; cancer growth and metastasis; viral infections, cancer therapy, acute lymphoblastic leukemia; in gliomas; neurologic diseases; treatment of cancer; neurodegenerative disorders; Alzheimer's Disease; Parkinson's Disorder; and hematopoietic disorders. The method includes contacting a test compound with a NOV polypeptide and determining if the test compound binds to said NOV polypeptide. Binding of the test compound to the NOV polypeptide indicates the test compound is a modulator of activity, or of latency or predisposition to the aforementioned disorders or syndromes.

Also within the scope of the invention is a method for screening for a modulator of activity, or of latency or predisposition to a disorders or syndromes listed above by administering a test compound to a test animal at increased risk for the aforementioned disorders or syndromes. The test animal expresses a recombinant polypeptide encoded by a NOV nucleic acid. Expression or activity of NOV polypeptide is then measured in the test animal, as is expression or activity of the protein in a control animal which recombinantly-expresses NOV polypeptide and is not at increased risk for the disorder or syndrome. Next, the expression of NOV polypeptide in both the test animal and the control animal is compared. A change in the activity of NOV polypeptide in the test animal relative to the control animal indicates the test compound is a modulator of latency of the disorder or syndrome.

In yet another aspect, the invention includes a method for determining the presence of or predisposition to a disease associated with altered levels of a NOV polypeptide, a NOV nucleic acid, or both, in a subject (e.g., a human subject). The method includes measuring the amount of the NOV polypeptide in a test sample from the subject and comparing the amount of the polypeptide in the test sample to the amount of the NOV polypeptide present in a control sample. An alteration in the level of the NOV polypeptide in the test sample as compared to the control sample indicates the presence of or predisposition to a disease in the subject. Preferably, the predisposition including those listed in the preferred embodiment. above.

In a further aspect, the invention includes a method of treating or preventing a pathological condition associated with a disorder in a mammal by administering to the subject a NOV polypeptide, a NOV nucleic acid, or a NOV-specific antibody to a subject (e.g., a human subject), in an amount sufficient to alleviate or prevent the pathological condition.

Unless otherwise defined, all technical and scientific terms used herein have the same meaning as commonly understood by one of ordinary skill in the art to which the invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative purposes only, and not intended to be limiting in any manner. Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their polypeptides. The sequences are collectively referred to as “NOV nucleic acids” or “NOV polynucleotides” and the corresponding encoded polypeptides are referred to as “NOV polypeptides” or “NOV proteins”. Unless indicated otherwise, “NOV” is meant to refer to any of the novel sequences disclosed herein. The below chart provides a summary of the NOV nucleic acids and their encoded polypeptides.

Sequences and Corresponding SEQ ID Numbers

| | | SEQ ID | SEQ ID | |
|---|---|---|---|---|
| | | number of | number of | |
| | | nucleic | encoded | |
| NOV | | acid | amino acid | Putative |
| number | clone | sequence | sequence | function |
| 1a | AC015663_A | 1 | 2 | Immuno- |
| | | | | regulation |
| 1b | | 3 | 4 | Immuno- |
| | | | | regulation |
| 2 | AF038458_A | 5 | 6 | cell |
| | | | | surface |
| | | | | receptor |
| | | | | and |
| | | | | signaling |

NOV1

A NOV1 nucleic acid sequence according to the invention includes a nucleic acid encoding a polypeptide related to the immunoregulatory protein interferon omega-1. TABLE 1 includes representative examples of NOV1 nucleic acid sequences and the included polypeptides. The disclosed NOV1b nucleic acid (SEQ ID NO:3) is 610 nucleotides in length and differs from the disclosed NOV1a nucleic acid (SEQ ID NO:1) by the insertion of 135 nucleotides following position 73 in the NOV1a sequence. The ORF start and stop codons are indicated in bold in TABLE 1. The sequences are listed below.

TABLE 1
Nucleotide sequences encoding the interferon omega-1 like proteins according to
the invention. The start and stop codons are in bold type.
NOV1a

| | | |
|---|---|---|
| ACCA ATG GTC TCC TTG CTG GTG GCA TTG GTG ATG ATC TCC TGC CAC ATC | 49 | (SEQ ID NO:1) |
| TAT TCC CTT TTC TGC GAC CTG CCT AAA GCT CAG GTG ATT TCT GCC CTC | 97 | |
| CAT AAG ATG CAC CAG CAG ATC TTC AGC CTC TTT TTA CAC AAG GGC TTG | 145 | |
| TCT GAT GCT TGG AAT AGG GCC TTC CTG GAC AAA CTC CAG ACT GGA TTT | 193 | |
| CAT CAG CAG CTG GAA GAC CTG GAG ACC TGC TTT GGT ATA GAG GAT GGG | 241 | |
| AAG CAA GAG TCT GCC CTG GAA ATT GAG GGC CCT ACA CTG GCC ATA AAG | 289 | |
| AGG TAC TTC CAG GGA GTA CAT TTC TTC TTG AAA GAG AGG AAA TTC AGG | 337 | |
| AAC TGT ACC TGG GAG GTT GTC GTA ATG GTA AAG GGA TTT TTC TTA AGC | 385 | |
| ACA AAA CTT CAA GAA AAA GAG AAC AGA AGA AAA GAG AAC TGC AAA AAA | 433 | |
| AAT CTG GAA AAG GTA ATC TAT TTA GCA GAA GAG TGA AAGCTG | 475 | |

Amino acid sequences of the interferon omega-1 like proteins according to the
invention.
NOV1a peptide

| | | |
|---|---|---|
| Met Val Ser Leu Leu Val Ala Leu Val Met Ile Ser Cys His Ile Tyr | | (SEQ ID NO:2) |
| 1 5 10 15 | | |
| Ser Leu Phe Cys Asp Leu Pro Lys Ala Gln Val Ile Ser Ala Leu His | | |
| 20 25 30 | | |
| Lys Met His Gln Gln Ile Phe Ser Leu Phe Leu His Lys Gly Leu Ser | | |
| 35 40 45 | | |
| Asp Ala Trp Asn Arg Ala Phe Leu Asp Lys Leu Gln Thr Gly Phe His | | |
| 50 55 60 | | |
| Gln Gln Leu Glu Asp Leu Glu Thr Cys Phe Gly Ile Glu Asp Gly Lys | | |
| 65 70 75 80 | | |
| Gln Glu Ser Ala Leu Glu Ile Glu Gly Pro Thr Leu Ala Ile Lys Arg | | |
| 85 90 95 | | |
| Tyr Phe Gln Gly Val His Phe Phe Leu Lys Glu Arg Lys Phe Arg Asn | | |
| 100 105 110 | | |
| Cys Thr Trp Glu Val Val Val Met Val Lys Giy Phe Phe Leu Ser Thr | | |
| 115 120 125 | | |
| Lys Leu Gln Glu Lys Glu Asn Arg Arg Lys Glu Asn Cys Lys Lys Asn | | |
| 130 135 140 | | |
| Leu Glu Lys Val Ile Tyr Leu Ala Glu Glu | | |
| 145 150 | | |
| NOV1b | | |
| ACCA ATG GTC TCC TTG CTG GTG GCA TTG GTG ATG ATC TCC TGC CAC ATC | 49 | (SEQ ID NO:3) |
| TAT TCC CTT TTC TGC GAC CTG CCT NNN NNN NNN NNN NNN NNN NNN NNN | 97 | |
| NNN NNN NNN NNN NNN NNN NNN NNN NNN NNN NNN NNN NNN NNN NNN NNN | 145 | |
| NNN NNN NNN NNN NNN NNN NNN NNN NNN NNN NNN NNN NNN NNN NNN NNN | 193 | |
| NNN NNN NNN NNN NNN AAA GCT CAG GTG ATT TCT GCC CTC CAT AAG ATG | 241 | |
| CAC CAG CAG ATC TTC AGC CTC TTT TTA CAC AAG GGC TTG TCT GAT GCT | 289 | |
| TGG AAT AGG GCC TTC CTG GAC AAA CTC CAG ACT GGA TTT CAT CAG CAG | 337 | |
| CTG GAA GAC CTG GAG ACC TGC TTT GGT ATA GAG GAT GGG AAG CAA GAG | 385 | |
| TCT GCC CTG GAA ATT GAG GGC CCT ACA CTG GCC ATA AAG AGG TAC TTC | 433 | |
| CAG GGA GTA CAT TTC TTC TTG AAA GAG AGG AAA TTC AGG AAC TGT ACC | 481 | |
| TGG GAG GTT GTC GTA ATG GTA AAG GGA TTT TTC TTA AGC ACA AAA CTT | 529 | |
| CAA GAA AAA GAG AAC AGA AGA AAA GAG AAC TGC AAA AAA AAT CTG GAA | 577 | |
| AAG GTA ATC TAT TTA GCA GAA GAG TGA AAGCTG | 610 | |
| NOV1b peptide | | |
| Met Val Ser Leu Leu Val Ala Leu Val Met Ile Ser Cys His Ile Tyr | | (SEQ ID NO:4) |
| 1 5 10 15 | | |
| Ser Leu Phe Cys Asp Leu Pro Xaa Xaa Xaa Xaa Xaa Xaa Xaa Xaa Xaa | | |
| 20 25 30 | | |
| Xaa Xaa Xaa Xaa Xaa Xaa Xaa Xaa Xaa Xaa Xaa Xaa Xaa Xaa Xaa Xaa | | |
| 35 40 45 | | |
| Xaa Xaa Xaa Xaa Xaa Xaa Xaa Xaa Xaa Xaa Xaa Xaa Xaa Xaa Xaa Xaa | | |
| 50 55 60 | | |
| Xaa Xaa Xaa Xaa Lys Ala Gln Val Ile Ser Ala Leu His Lys Met His | | |
| 65 70 75 80 | | |
| Gln Gln Ile Phe Ser Leu Phe Leu His Lys Gly Leu Ser Asp Ala Trp | | |
| 85 90 95 | | |
| Asn Arg Ala Phe Leu Asp Lys Leu Gln Thr Gly Phe His Gln Gln Leu | | |
| 100 105 110 | | |
| Glu Asp Leu Glu Thr Cys Phe Gly Ile Glu Asp Gly Lys Gln Glu Ser | | |
| 115 120 125 | | |
| Ala Leu Glu Ile Glu Gly Pro Thr Leu Ala Ile Lys Arg Tyr Phe Gln | | |
| 130 135 140 | | |
| Gly Val His Phe Phe Leu Lys Glu Arg Lys Phe Arg Asn Cys Thr Trp | | |
| 145 150 155 160 | | |
| Glu Val Val Val Met Val Lys Gly Phe Phe Leu Ser Thr Lys Leu Gln | | |
| 165 170 175 | | |
| Glu Lys Glu Asn Arg Arg Lys Glu Asn Cys Lys Lys Asn Leu Glu Lys | | |
| 180 185 190 | | |
| Val Ile Tyr Leu Ala Glu Glu | | |
| 195 | | |

The insertion of nucleotides in the middle of the ORF NOV1b nucleic acid relative to the NOV1a nucleic acid leads to an insertion of 45 amino acids in the NOV1b polypeptide (SEQ ID NO:4) at position 23 relative to the NOV1a polypeptide (SEQ ID NO:2), which is 154 amino acids in length.

Homology

Based on a substantial degree of homology to various members of the interferon gene family, NOV1 can be characterized as a member of the interferon family. Using BLASTX comparison and translation of the ORF, the disclosed NOV1a polypeptide (SEQ ID NO:2) has 54 of 112 amino acids identical to, and 79 of 112 amino acids positive to, a 195 amino acid segment of Bos Taurus (bovine) interferon omega-1 precursor (SWISSPROT-ACC:P07352). Using BLASTN nucleotide comparison, the NOV1a nucleic acid sequence has 277 nucleotides (75%) identical to a 365 nucleotide segment of Equus Caballas horse, interferon-omega-2 (GenBank ID: HRSIFN7 ACC:M14545). In this comparison, the Query is NOV1a and the subject is GenBank Accession No. M14545 (SEQ ID NO:7). This 75% sequence identity is illustrated below.

Alignment of NOV1a nucleic acid with Horse interferon-omega-2 (GenBank ID
HRS1FN1/all:M14545)
Top Previous Match Next Match
Length = 802
Plus Strand HSPs:
Score = 943 (141.5 bits), Expect = 1.8e − 36, P = 1.8e − 36
Identities = 277/365 (75%) , Positives = 277/365 (75%) , Strand = Plus/Plus

| | | | | |
|---|---|---|---|---|
| Query: | 69 | TGCCTAAAGCTCAGGTGATTTCTGCCCTCCATAAGATGCACCAGCAGATCTTCAGCCTCT | 128 | (SEQ ID NO:1) |
| | | \| \|\| \| \|\| \|\|\|\| \|\|\|\| \|\|\|\|\|\| \|\|\|\|\|\| \|\|\|\|\|\|\|\|\|\|\|\|\|\|\|\|\|\|\|\| | | |
| Sbjct: | 340 | TCCCAGAGGCCCAGGCCGCGTCTGTCCTCCACGAGATGCTCCAGCAGATCTTCAGCCTCT | 399 | (SEQ ID NO:7) |
| Query: | 129 | TTTTACACA-AGGGCTTGTCTGATGCTTGGAATAGGGCCTTCCTGGACAAACTCCAGACT | 187 | |
| | | \| \|\|\|\|\| \|\| \|\|\| \|\|\|\|\| \|\|\| \|\|\|\|\| \| \| \|\| \|\|\|\|\|\|\|\|\| \|\|\|\|\| \|\| | | |
| Sbjct: | 400 | TCC-ACACAGAGCGCTCGTCTGCTGCCTGGAACACGACCCTCCTGGACGAACTCTGCACG | 458 | |
| Query: | 188 | GGATTTCATCAGCAGCTGGAAGACCTGGAGACCTGCTTTGGTATAGAGGATGGGA-AGCA | 246 | |
| | | \|\|\| \| \| \|\| \|\|\|\|\|\|\|\|\|\|\|\|\|\|\|\|\|\| \|\|\|\|\| \|\|\|\|\| \|\| \|\|\|\|\|\|\| \|\| \| | | |
| Sbjct: | 459 | GGACTCCTTCGGCAGCTGGAAGACCTGGACACCTG-TTTGGAGCAGGAGATGGGAGAGGA | 517 | |
| Query: | 247 | AGAGTCTGCCCTGGAAATTGAGGGCCCTACACTGGCCATAAAGAGGTACTTCCAGGGAGT | 306 | |
| | | \|\|\| \|\|\|\|\|\|\|\|\|\| \|\| \|\| \| \|\|\|\|\|\|\|\|\|\|\|\|\|\| \| \|\|\|\|\|\|\|\|\|\|\|\|\| \|\|\| \| | | |
| Sbjct: | 518 | AGAATCTGCCCTGGGAACTGTGCGCCCTACACTGGCCGTGAAGAGGTACTTCCGGGGGAT | 577 | |
| Query: | 307 | ACATTTCTTCTTGAAAGAGAGGAAATTCAGGAACTGTACCTGGGAGGTTGTCGTAATGGT | 366 | |
| | | \|\|\| \|\|\| \| \|\|\|\|\|\|\|\|\| \|\|\|\|\| \|\|\| \|\|\|\|\| \|\|\|\|\|\|\|\| \|\|\|\|\| \|\|\|\|\| | | |
| Sbjct: | 578 | CCATCTCTACCTGAAAGAGAAGAAATACAGTGACTGTGCCTGGGAGATTGTCCGAATGGA | 637 | |
| Query: | 367 | AA--AGG-GATT-TT-TCTTAAGCA-CAAAACTTCAAGAAAAAG-A-GAACAGAAGAAAA | 418 | |
| | | \|\| \| \| \|\|\| \|\| \|\|\|\| \| \|\| \|\|\|\| \|\| \|\|\|\| \|\| \| \|\|\| \|\|\|\| \| | | |
| Sbjct: | 638 | AATCATGAGATCCTTCTCTTCATCAGCAAACCTGCAAGGAAGGTTAAGAAT-GAAGGATG | 696 | |
| Query: | 419 | GAGAACTG | 426 | |
| | | \|\|\|\| \|\|\| | | |
| Sbjct: | 697 | GAGACCTG | 704 | |

Using the ClustalW alignment algorithm as shown in TABLE 2, a multiple alignment with similar proteins shows comparable degrees of homology between the NOV1a polypeptide sequence, human, and horse interferon omega-1 precursor. Based on its relatedness to interferon omega-1, the NOV1a protein is a novel member of the interferon gene family.

TABLE 2
Alignment of a NOV1 amino acid sequence with horse (PID g124499) and human (PID g400061) interferon omega1-precursor

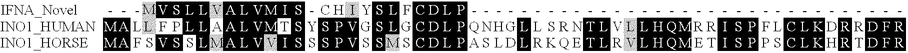

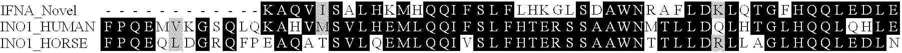

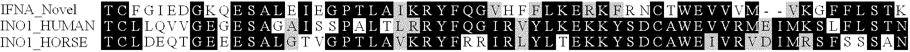

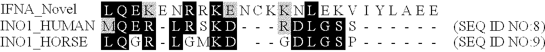

Additionally, domain searches provide further evidence that NOV 1a is a member of the interferon family, since it contains domains which are highly conserved among the interferon family. NOV1a shares conserved domains as illustrated below. The E-value demonstrates that the likelihood of NOV1a sharing the conserved interferon domain is not due to chance alone, but is an accurate reflection of the actual conservation of the domain. The conserved domains with their respective E-values are illustrated below.

| | | Score | E |
|---|---|---|---|
| Sequences producing significant alignments: | | (bits) | value |
| gnl\|Pfam\|pfam00143 | Interferon, Interferon | 110 | 6e-26 |
| | alpha/beta domain | | |
| gnl\|Smart\|IFabd | Interferon alpha, beta and delta.; | 97.9 | 3e-22 |
| | Interferons produce antivir . . . | | |

The first domain (gnl|Pfam|pfam00143) is aligned with NOV1a below. In this comparison, the Query is NOV1a and the subject is pfam00143 (SEQ ID NO:10).

gnl|Pfam|pfam00143, interferon, Interferon alpha/beta domain.

Length = 194

| | | | | |
|---|---|---|---|---|
| | | Score = 110 bits (272), Expect = 6e − 26 | | |
| Query: | 22 | LPKAQVISALHKMHQQIFSLFLHKGLSDA-WNRAFLDKLQTGFHQQLEDLETCFGIEDGK | 80 | (SEQ ID NO:10) |
| Sbjct: | 71 | SHKAQALSVVHVTNQKIFHFFCTEASSSAAWNTTLLEEFCTGLDRQLTRLEACVLQEVEE | 130 | |
| Query: | 81 | QESALEIEGPT---LAIKRYFQGVHFFLKERKFRNCTWEVV---VMVKGFFLSTKLQ | 131 | |
| Sbjct: | 131 | GEAPLTNEDIHPEDSILPNYFQRLSLYLQEKKYSPCAWEIVRAEIMRSLYYSSTALQ | 187 | |

A further database search reveals the following top four related proteins which contain the above interferon family domain. These results are illustrated below.

Top 4 related proteins with interferon alphalbeta domain:

| | |
|---|---|
| gi\|547724 INTERFERON ALPHA PRECURSOR (IFN-ALPHA) | (SEQ ID NO:11) |
| gi\|585317 INTERFERON DELTA-1 PRECURSOR | (SEQ ID NO:12) |
| gi\|124502 INTERFERON OMEGA-2 PRECURSOR (INTERFERON ALPHA-II-2) | (SEQ ID NO:13) |
| gi\|400061 INTERFERON OMEGA-1 PRECURSOR (INTERFERON ALPHA-Il-1) | (SEQ ID NO:14) |

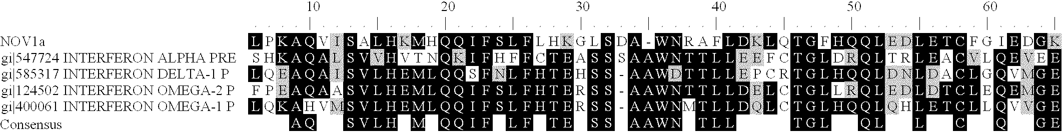

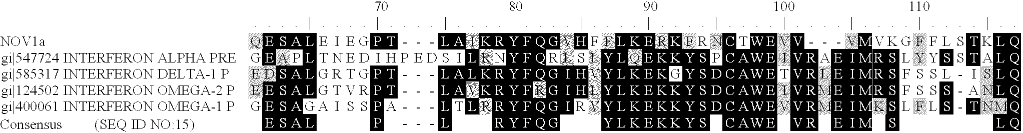

The second conserved domain, interferon alpha, beta, and delta, is aligned with NOV1a below. In this comparison, the Query is NOV1a and the subject is gnl|Smart|IFabd. The small E-value demonstrates that the conservation is not due to chance alone. This alignment is shown below.

Gnl|Smart|IFabd, Interferon alpha, beta and delta.; Interferons produce antiviral and antiproliferative responses in cells. They are classified into five groups, all of them related but gamma-interferon.

| | | Length = 118 | |
|---|---|---|---|
| | | Score = 97.9 bits (240), Expect = 3e − 22 | |
| Query: | 21 | DLPKAQVISALHKMHQQIFSLFLHKGLSDAWNRAFLDKLQTGFHQQLEDLETCFGIEDGK | 80 |
| Sbjct: | 13 | QFQKEDAALTIYEMLQNTFATFRQDSSSTGWNETTVENLLANVYHQINHLKTVLEEKLEK | 72 |
| Query: | 81 | QESALEIEGPTLAIKRYFQGVHFFLKERKFRNCTWEVV | 118 |
| Sbjct: | 73 | EDFTRGKLMSSLHLKRYYGRILHYLKAKEYSHCAWTIV | 110 |

A further database search reveals the following top four related proteins which contain the interferon alpha, beta, and delta domain. These results are shown below.

Top 4 related proteins with interferon alpha, beta, and delta domain:

gi|3318960|pdb|1AU1|A Chain A, Human Interferon-Beta Crystal Structure
gi|2624437|pdb|1RH2|A Chain A, Recombinant Human Interferon-Alpha 2b
gi|124432 INTERFERON ALPHA-1 PRECURSOR
gi|2147609 interferon-omega20 - rabbit

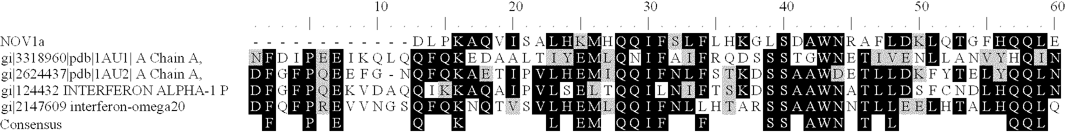

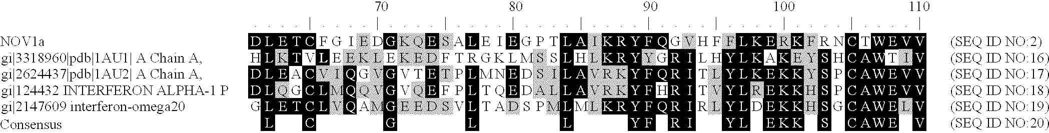

Domain searches provide evidence that NOV1b is also a member of the interferon family since it contains domains which are highly conserved among members of the interferon family. NOV1 b shares conserved domains as illustrated below. The small E-value demonstrates that the probability of NOV1b sharing the conserved interferon domain is not due to chance alone, but rather is an accurate reflection of the actual conservation of the domain. The conserved domains are illustrated below.

| | | Score | E |
|---|---|---|---|
| Sequences producing significant alignments: | | (bits) | value |
| gnl\|Pfam\|pfam00143 | Interferon, Interferon | 113 | 9e-27 |
| | alpha/beta domain | | |
| gnl\|Smart\|IFabd | Interferon alpha, beta and delta.; | 97.5 | 6e-22 |
| | Interferons produce antivir . . . | | |

The first domain, interferon alpha/beta (pfam00143) is aligned with NOV1b below. In this comparison, the Query is NOV1b and the subject is pfam00143 (SEQ ID NO:10).

gnl|Pfam|pfam00143, interferon, Interferon alpha/beta domain.

Length = 194

| | | | |
|---|---|---|---|
| | | Score = 113 bits (280) , Expect = 9e − 27 | |
| Query: | 2 | VSLLVALVMISCH-IYSLFCDLPXXXXXXXXXXXXXXXXXXXXXXXXXXXXXXXXXXXXX | 60 |
| Sbjct: | 5 | SSFLVALVALGCNSVCSLGCDLPQTHGLLNRRALTLLGQMRRLPASSCQKDRNDFAFPQD | 64 |
| Query: | 61 | XXXXXXXXKAQVISALHKMHQQIFSLFLHKGLSDA-WNRAFLDKLQTGFHQQLEDLETCF | 119 |
| Sbjct: | 65 | VFGGDQSHKAQALSVVHVTNQKIFHFFCTEASSSAAWNTTLLEEFCTGLDRQLTRLEACV | 124 |
| Query: | 120 | GIEDGKQESALEIEGPT---LAIKRYFQGVHFFLKERKFRNCTWEVV---VMVKGFFLST | 173 |
| Sbjct: | 125 | LQEVEEGEAPLTNEDIHPEDSILRNYFQRLSLYLQEKKYSPCAWEIVRAEIMRSLYYSST | 184 |
| Query: | 174 | KLQ | 176 |
| Sbjct: | 185 | ALQ | 187 |

A further database search reveals the following Top 4 related proteins which contain the interferon alpha/beta domain. The results are illustrated below.

Top 4 related proteins with interferon alpha/beta domain:

gi|547724 INTERFERON ALPHA PRECURSOR (IFN-ALPHA)
gi|124431 INTERFERON ALPHA-1 PRECURSOR
gi|124502 INTERFERON OMEGA-2 PRECURSOR (INTERFERON ALPHA-II-2)
gi|400061 INTERFERON OMEGA-1 PRECURSOR (INTERFERON ALPHA-II-1)

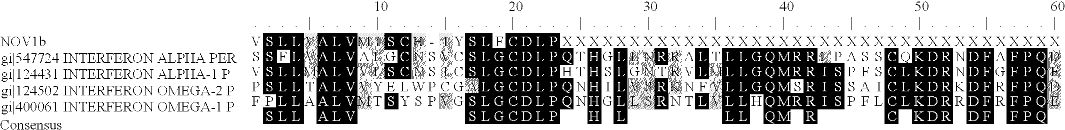

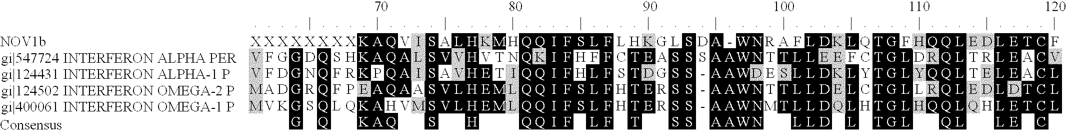

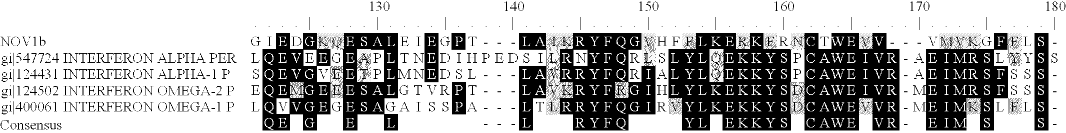

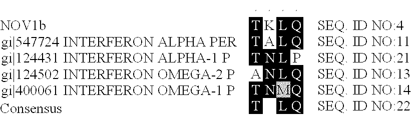

The second conserved domain interferon alpha, beta, and delta, is aligned with NOV1b below. In this comparison, the Query is NOV1b and the subject is gnl|Smart|IFabd. The small E-value demonstrates that the conservation is not due to chance alone. This alignment is shown below.

gnl|Smart|IFabd, Interferon alpha, beta and delta.; Interferons produce antiviral and antiproliferative responses in cells. They are classified into five groups, all of them related but gamma-interferon.

Length = 118
Score = 97.5 bits (239),
Expect = 6e-22

| | | | |
|---|---|---|---|
| Query: | 69 | KAQVISALHKMHQQIFSLFLHKGLSDAWNRAFLDKLQTGFHQQLEDLETCFGIEDGKQES | 128 |
| Sbjct: | 16 | KEDAALTIYEMLQNIFAIFRQDSSSTGWNETIVENLLANVYHQINHLKTVLEEKLEKEDF | 75 |
| Query: | 129 | ALEIEGPTLAIKRYFQGVHFFLKERKFRNCTWEVV | 163 |
| Sbjct: | 76 | TRGKLMSSLHLKRYYGRILHYLKAKEYSHCAWTIV | 110 |

A further database search reveals the following Top 4 related proteins which contain the interferon alpha, beta, and delta domain. The results are illustrated below.

Top 4 related proteins with interferon alpha, beta, and delta domain:

[image]

Based on the above alignments demonstrating significant homology, NOV1 is a member of the interferon family.

The Type I interferons (IFN-alpha, IFN-beta, and IFN-omega) are a family of closely related cytokines that have antiviral and immunostimulatory properties. They bind to the type I IFN receptor (IFNAR) and elicit signaling events including activation of the Jak/Stat and IRS pathways. There has been prolonged debate regarding the different interferon-alpha subtypes: with some authorities suggesting that the different interferons have essentially similar properties but others arguing that there are significant differences between them. Recent work has shown that the various interferon-alpha subtypes can interact with the interferon receptor components in different ways and can activate a number of different signalling pathways. Recent studies on the immunomodulatory properties of the Type I interferons indicate that there are profound differences between the subtypes (Hajnicka V, Fuchsberger N, Liptakova H, Stancek D, Kontsek P. Acta Virol 1996 September;40(4):221-2; Tiefenthaler M, Geisen F, Schirmer M, Konwalinka G J Interferon Cytokine Res 1997 June;17(6):327-9)

Before this invention a single functional gene in the human genome was known to code for interferon omega (IFN-omega), a monomeric glycoprotein distantly related in structure to IFN-alpha and IFN-beta, but unrelated to IFN-gamma. IFN-omega is secreted by virus-infected leukocytes as a major component of human leukocyte interferon. The human class I IFN receptor complex, which mediates the biological activity of IFN-alpha and IFN-beta also binds IFN-omega. Potent antiviral activity against several DNA and RNA viruses has been demonstrated. IFN-omega inhibits proliferation of a variety of tumor cell lines in vitro. The protein stimulates natural killer cell activity, enhances expression of major histocompatibility complex class I (but not class II) antigens and inhibits proliferation of lymphocytes stimulated with mitogens or allogeneic cells. IFN-omega is unrelated to IFN-alpha, -beta and -gamma in its antigenic properties, as it does not cross-react with antisera or monoclonal antibodies in immunoassays or antiviral neutralization bioassays. Antibodies induced in patients by long-term IFN-alpha 2 therapy that block IFN-alpha 2 activity do not inactivate IFN-omega.

Human interferon-omega has been shown to suppresses hepatitis B surface antigen production in human hepatoma cell line (Hajnicka V, Fuchsberger N, Liptakova H, Stancek D, Kontsek P. Acta Virol 1996 September;40(4):221-2), and IFN-omega may prove an effective treatment for chronic myelogenous leukemia patents who become resistant to IFN-alpha 2 because of the formation of neutralizing antibodies (Tiefenthaler M, Geisen F, Schirmer M, Konwalinka G J Interferon Cytokine Res 1997 June;17(6):327-9). The bovine IFN-omega 1, which this invention has homology with, is biologically active and secreted at high levels in the yeast Pichia pastoris (Rodriguez M, Martinez V, Alazo K, Suarez M, Redondo M, Montero C, Besada V, de la Fuente J. J Biotechnol Feb. 5, 1998;60(1-2):3-14).

Henco et al. (1985) compiled partial maps of the immunoregulatory interferon gene cluster located on 9p21. These maps showed that members of the 2 main families of genes in the IFN superfamily, interferon-alpha (147660) and interferon-omega (IFNW), are interspersed. Olopade et al. (1992) studied the deletions of the short arm of chromosome 9 frequently observed in acute lymphoblastic leukemia and in gliomas. These deletions often include the entire interferon gene cluster, which comprises about 26 IFNA, IFNW, and IFNB1 (147640) genes, as well as the gene for methylthioadenosine phosphorylase (MTAP; 156540). By comparing microscopic deletions with the genes lost at the molecular level, Olopade et al. (1992) determined the order of these genes on 9p to be tel--IFNB1--IFNA/IFNW cluster--MTAP--cen. In a few cell lines and in primary leukemia cells, they observed deletions that had breakpoints within the IFN gene cluster and resulted in partial loss of the IFN genes. These partial deletions allowed them to determine the order of some genes or groups of genes in the IFNA/IFNW gene cluster. From their deletion analysis, Olopade et al. (1992) deduced the following order of the IFN gene on 9p:pter--IFNB1--(IFNW1, IFNA21)--IFNWP15--IFNA4--IFNW9--IFNA7--IFNA10--IFNWP18--IFNAP16--IFNA17--IFNA14--(IFNA22, IFNA5, IFNAP20, IFNA6, IFNA13, IFNA2)--(IFNA8, IFNW2, IFNWP19, IFNA1)--MTAP--cen. The genes within the large linkage group are arranged in tandem with their 3-prime end pointing toward the telomere of the short arm. Thus, at least 2 functional interferon-omega genes, IFNW1 and IFNW2, were mapped and several IFNW pseudogenes, e.g., IFNWP15, were localized.

The discovery of molecules related in the interferons satisfies a need in the art by providing new diagnostic or therapeutic compositions useful in the treatment of disorders associated with alterations in the expression of members of the interferon famly. Based on its relatedness to interferon omega-1, nucleic acids, polypeptides, antibodies, and other compositions of the present invention will be useful therapeutics for immunotherapy, viral infections, cancer therapy, neurologic diseases, and especially—acute lymphoblastic leukemia and gliomas. Also, based on its relatedness to interferon omega-1, expression of nucleic acids or polypeptides of the present invention can be used to identify virally infected cells. The invention may also be used in assays to identify the inhibitition of proliferation of lymphocytes stimulated with mitogens or allogeneic cells. The NOV1 protein can be used to stimulate natural killer cell activity and enhance the presentation of major histocompatibility complex antigens.

NOV2

A NOV2 nucleic acid according to the invention includes nucleic acids encoding a novel transmembrane polypeptide. An example of this nucleic acid along with the encoded polypeptide are presented in TABLE 5. The disclosed nucleic acid (SEQ ID NO:5) is 1887 nucleotides in length and contains an ORF that begins with an ATG initiation codon at nucleotides 1-3 and ends with a TAG stop codon at nucleotides 1885-1887. The representative ORF includes a 628 amino acid polypeptide (SEQ ID NO:6), which is presented in TABLE 5.

TABLE 5

Nucleotide sequences encoding the novel transmembrane protein according
to the invention. The start and stop codons are in bold type.

| | |
|---|---|
| ATGGCCATCCTCCCGTTGCTCCTGTGCCTGCTGCCGCTGGCCCCTGCCTCATCCCCACCCCAGTCAGCCACACCCAG | (SEQ ID NO:5) |
| CCCATGTCCCCGCCGCTGCCGCTGCCAGACACAGTCGCTGCCCCTAAGCGTGCTGTGCCCAGGGGCAGGCCTCCTGT | |
| TCGTGCCACCCTCGCTGGACCGCCGGGCAGCCGAGCTGCGGCTGGCAGACAACTTCATCGCCTCCGTGCGCCGCCGC | |
| GACCTGGCCAACATGACAGGCCTGCTGCATCTGAGCCTGTCGCGGAACACCATCCGCCACGTGGCTGCCGGCGCCTT | |
| CGCCGACCTGCGGGCCCTGCGTGCCCTGCACCTGGATGGCAACCGGCTGACCTCACTGGGCGAGGGCCAGCTGCGCG | |
| GCCTGGTCAACTTGCGCCACCTCATCCTCAGCAACAACCAGCTGGCAGCGCTGGCGGCCGGCGCCCTGGATGATTGT | |
| GCCGAGACACTGGAGGACCTCGACCTCTCCTACAACAACCTCGAGCAGCTGCCCTGGGAGGCCCTGGGCCGCCTGGG | |
| CAACGTCAACACGTTGGGCCTCGACCACAACCTGCTGGCTTCTGTGCCCGCCGGCGCTTTTTCCCGCCTGCACAAGC | |
| TGGCCCGGCTGGACATGACCTCCAACCGCCTGACCACAATCCCACCCGACCCACTCTTCTCCCGCCTGCCCCTGCTC | |
| GCCAGGCCCCGGGGCTCGCCCGCCTCTGCCCTGGTGCTGGCCTTTGGCGGGAACCCCCTGCACTGCAACTGCGAGCT | |
| GGTGTGGCTGCGTCGCCTGGCGCGGGAGGACGACCTCGAGGCCTGCGCGTCCCCACCTGCTCTGGGCGGCCGCTACT | |
| TCTGGGCGGTGGGCGAGGAGGAGTTTGTCTGCGAGCCGCCCGTGGTGACTCACCGCTCACCACCTCTGGCTGTGCCC | |
| GCAGGTCGGCCGGCTGCCCTGCGCTGCCGGGCAGTGGGGGACCCAGAGCCCCGTGTGCGTTGGGTGTCACCCCAGGG | |
| CCGGCTGCTAGGCAACTCAAGCCGTGCCCGCGCCTTCCCCAATGGGACGCTGGAGCTGCTGGTCACCGAGCCGGGTG | |
| ATGGTGGCATCTTCACCTGCATTGCGGCCAATGCAGCTGGCGAGGCCACAGCTGCTGTGGAGCTGACTGTGGGTCCC | |
| CCACCACCTCCTCAGCTAGCCAACAGCACCAGCTGTGACCCCCCGCGGGACGGGGATCCTGATGCTCTCACCCCACC | |
| CTCCGCTGCCTCTGCTTCTGCCAAGGTGGCCGACACTGGGCCCCCTACCGACCGTGGCGTCCAGGTGACTGAGCACG | |
| GGGCCACAGCTGCTCTTGTCCAGTGGCCGGATCAGCGGCCTATCCCGGGCATCCGCATGTACCAGATCCAGTACAAC | |
| AGCTCGGCTGATGACATCCTCGTCTACAGGATGATCCCGGCGGAGAGCCGCTCGTTCCTGCTGACGGACCTGGCGTC | |
| AGGCCGGACCTACGATCTGTGCGTGCTCGCCGTGTATGAGGACAGCGCCACGGGGCTCACGGCCACGCGGCCTGTGG | |
| GCTGCGCCCGCTTCTCCACCGAACCTGCGCTGCGGCCATGCGGGGCGCCGCACGCTCCCTTCCTGGGCGGCACGATG | |
| ATCATCGCGCTGGGCGGCGTCATCGTAGCCTCGGTACTGGTCTTCATCTTCGTGCTGCTAATGCGCTACAAGGTGCA | |
| CGGCGGCCAGCCCCCCGGCAAGGCCAAGATTCCCGCGCCTGTTAGCAGCGTTTGCTCCCAGACCAACGGCGCCCTGG | |
| GCCCCACGCCCACGCCCGCCCCGCCCGCCCCGGAGCCCGCGGCGCTCAGGGCCCACACCGTGGTCCAGCTGGACTGC | |
| GAGCCCTGGGGGCCCGGCCACGAACCTGTGGGACCCTAG | |

Amino acid sequences of the novel transmembrane
protein according to the invention.

| | |
|---|---|
| MAILPLLLCLLPLAPASSPPQSATPSPCPRRCRCQTQSLPLSVLCPGAGLLFVPPSLDRRAAELRLADNFIASV | (SEQ ID NO:6) |
| RRRDLANMTGLLHLSLSRNTIRHVAAGAFADLRALRALHLDGNRLTSLGEGQLRGLVNLRHLILSNNQLA | |
| ALAAGALDDCAETLEDLDLSYNNLEQLPWEALGRLGNVNTLGLDHNLLASVPAGAFSRLHKLARLDMTS | |
| NRLTTIPPDPLFSRLPLLARPRGSPASALVLAFGGNPLHCNCELVWLRRLAREDDLEACASPPALGGRYFWA | |
| VGEEEFVCEPPVVTHRSPPLAVPAGRPAALRCRAVGDPEPRVRWVSPQGRLLGNSSRARAFPNGTLELLVT | |
| EPGDGGIFTCIAANAAGEATAAVELTVGPPPPPQLANSTSCDPPRDGDPDALTPPSAASASAKVADTGPPTD | |
| RGVQVTEHGATAALVQWPDQRPIPGIRMYQIQYNSSADDILVYRMIPAESRSFLLTDLASGRTYDLCVLAV | |
| YEDSATGLTATRPVGCARFSTEPALRPCGAPHAPFLGGTMIIALGGVIVASVLVFIFVLLMRYKVHGGQPPG | |
| KAKIPAPVSSVCSQTNGALGPTPTPAPPAPEPAALRAHTVVQLDCEPWGPGHEPVGP | |

Homology

Based on a substantial degree of homology to a transmembrane protein, NOV2 can be characterized as a member of the transmembrane protein family. The disclosed NOV2 nucleic acid is identical to 1171 of 1758 (66%) nucleotides from a human on RNA for the KIAA1246 protein which is expressed in fetal brain (GenBank, Accession No. AB033072).

Using BLASTX comparison and translation of the representative ORF, the disclosed NOV2 protein has 313 amino acids identical to, and 402 amino acids positive to, a 581 amino acid segment of human KIAA1246 protein (TREMBLNEW-ACC:BAA86560). The NOV2 polypeptide has a strong signal peptide and is likely to be cleaved between amino acids 16 and 17. The NOV2 polypeptide also sorts to the plasma membrane or the ER with equal confidence. The homology of NOV2 nucleotide and polypeptide are shown below. In these alignments, NOV2 is the Query and KIAA1246 on RNA and protein are the subjects.

TABLE 6

Alignment of GENBANKNEW-ID:AB033072|acc:AB033072 Homo sapiens
mRNA for KIAA1246 protein, partial cds - Homo sapiens with a NOV2 nucleic acid
BLASTN:
>gb:GENBANKNEW-ID:AB033072|acc:AB033072 Homo sapiens mRNA
for KIAA1246 protein, partial cds - Homo sapiens, 3144 bp
Score = 2927 (439.2 bits), Expect = 1.0e-126, P = 1.0e-126
Identities = 1171/1758 (66%), Positives = 1171/1758 (66%), Strand = Plus / Plus

| | | | | |
|---|---|---|---|---|
| Query: | 78 | CCCATGTCCCCGCCGCTGCCGCTGCCAGACACAGTCGCTGCCCCTAAGCGTGCTGTGCCC | 137 | (SEQ ID NO:5) |
| | | \| \| \|\| \|\|\| \|\|\| \|\|\|\|\|\|\|\| \| \|\|\| \| \| \|\| \| \|\|\|\|\|\|\|\| | | |
| Sbjct: | 522 | CGCCTGCCCCAAGTACTGTGTCTGCCAGAATCTGTCTGAGTCACTGGGGACCCTGTGCCC | 581 | (SEQ ID NO:23) |
| Query: | 138 | AGGGGCAGGCCTCCTGTTCGTGCCACCCTCGC-TGGACCGCCGGGCAGCCGAGCTGCGGC | 196 | |
| | | \|\| \|\| \|\| \|\| \|\| \|\| \|\|\|\| \| \|\|\|\|\| \|\|\| \|\|\| \|\|\|\|\|\|\|\| \| | | |
| Sbjct: | 582 | CTCCAAGGGGCTGCTCTTTGTACC-CCCTGATATTGACCGGCGGACAGTGGAGCTGCGCC | 640 | |
| Query: | 197 | TGGCAGACAACTTCATCGCCTCCGTGCGCCGCCGCGACCTGGCCAACATGACAGGCCTGC | 256 | |
| | | \|\|\| \| \|\|\|\|\|\|\|\|\|\| \| \| \| \|\|\|\|\|\| \|\|\| \| \|\|\|\|\|\|\|\|\|\|\| \|\| \|\|\| | | |
| Sbjct: | 641 | TGGGCGGCAACTTCATCATCCACATCAGCCGCCAGGACTTTGCCAACATGACGGGGCTGG | 700 | |
| Query: | 257 | TGCATCTGAGCCTGTCGCGGAACACCATCCGCCACGTGGCTGCCGGCG-CCTTCGCCGAC | 315 | |
| | | \|\| \| \|\|\|\| \|\|\|\|\|\| \|\|\|\|\|\|\|\|\|\|\| \|\|\|\|\| \| \| \|\|\| \|\|\|\| \|\|\| | | |
| Sbjct: | 701 | TGGACCTGACCCTGTCCAGGAACACCATCAGCCACATC-CAGCCCTTTTCCTTTCTGGAC | 759 | |
| Query: | 316 | CTGCGGGC-CCTGCGTGCCCTGCACCTGGATGGCAACCGGCTGACCTCACTGGGCGAGGG | 374 | |
| | | \|\| \|\| \| \|\|\| \|\| \|\|\|\|\|\|\| \|\| \|\| \|\|\|\| \|\|\|\|\|\| \| \|\| \|\| \|\|\|\| | | |
| Sbjct: | 760 | CT-CGAGAGCCTCCGCTCCCTGCATCTTGACAGCAATCGGCTGCCAAGCCTTGGGGAGGA | 818 | |
| Query: | 375 | CCAGCTGCGCGGCCTGGTCAACTTGCGCCACCTCATCCTCAGCAACAACCAGCTGGCAGC | 434 | |
| | | \| \|\| \|\| \|\|\|\|\|\|\|\|\|\|\|\| \|\|\| \|\|\|\|\| \|\|\| \| \| \|\|\|\|\|\|\|\|\|\|\|\|\|\| \| | | |
| Sbjct: | 819 | CACCCTCCGGGGCCTGGTCAACCTGCAGCACCTTATCGTGAACAACAACCAGCTGGGCG- | 877 | |
| Query: | 435 | GC-TGGCGGCCGGCGCCCTGGATGATTGTGCCGA-GACACTGGAGGACCTCGACCTCTCC | 492 | |
| | | \|\| \| \|\| \| \| \|\| \| \|\| \|\| \| \| \| \| \|\|\|\| \|\|\|\|\|\|\| \|\| \|\|\|\|\|\|\|\|\| | | |
| Sbjct: | 878 | GCATCGCAGATGAGGCTTTTGAGGACT-TCCTGCTGACATTGGAGGATCTGGACCTCTCC | 936 | |
| Query: | 493 | TACAACAACCTCGAGCAGCTGCCCTGGGAGGCCCTGGGCCGCCTGGGCAACGTCAACACG | 552 | |
| | | \|\|\|\|\|\|\|\|\|\|\|\| \| \|\|\|\|\| \|\|\|\|\| \|\| \|\| \| \|\|\| \|\|\| \|\|\|\| \|\| \|\| \| | | |
| Sbjct: | 937 | TACAACAACCTCCATGGCCTGCCGTGGGACTCCGTGCGACGCATGGTCAACCTCCACCAG | 996 | |
| Query: | 553 | TTGGGCCTCGACCACAACCTGCTGGCTTCTGTGCCCGCCGGCGCTTTTTCCCGCCTGCAC | 612 | |
| | | \|\| \|\|\|\| \|\|\|\|\|\|\|\|\|\|\|\|\|\|\|\| \| \| \|\|\| \|\|\| \| \|\|\| \| \|\|\|\|\|\| | | |
| Sbjct: | 997 | CTGAGCCTGGACCACAACCTGCTGGATCACATCGCCGAGGGCACCTTTGCAGACCTGCAG | 1056 | |
| Query: | 613 | AAGCTGGCCCGGCTGGACATGACCTCCAACCGCCTGACCACAATCCCACCCGA-CCCACT | 671 | |
| | | \|\| \|\|\|\|\|\|\|\| \|\|\|\|\| \| \|\|\|\|\|\|\|\| \|\| \|\|\| \| \| \|\| \|\| \|\| \|\|\|\| \| | | |
| Sbjct: | 1057 | AAACTGGCCCGCCTGGATCTCACCTCCAATCGGCTGCAGAAGCTGCCCCCTGATCCCA-T | 1115 | |
| Query: | 672 | CTTCTCCCGCCTGCCCCTGCT-CGCCAGGCCCCGGGGCTCGCCC-GCCTCTGCCCTGGTG | 729 | |
| | | \|\|\| \|\|\|\|\| \| \|\|\| \|\|\| \|\| \| \| \| \| \| \| \|\|\| \| \|\|\|\| \|\| | | |
| Sbjct: | 1116 | CTTTGCCCGC-T-CCCAGGCTTCGGCTTTGACAGCCACACCCTTTGCCCCA-CCCTTGTC | 1172 | |
| Query: | 730 | CTGGCCTTTGGCGGG-AACCCCCTGCACTGCAACTGCGAGCTGGTGTGGCTGCGTCGCCT | 788 | |
| | | \|\| \|\|\| \| \|\|\| \|\|\|\|\| \|\| \|\|\|\|\|\|\|\| \|\| \|\|\|\|\| \| \|\|\|\|\|\|\|\| \| \|\| | | |
| Sbjct: | 1173 | CTTTAGTTTTGGGGGTAACCCACTTCACTGCAATTGTGAGCTTCTCTGGCTGCGGAGGCT | 1232 | |
| Query: | 789 | GGCGCGGGAGGACGACCTCGAGGCCTGCGCGTCCCCACCTGCTCTGGGCGGCCGCTACTT | 848 | |
| | | \| \|\|\|\|\|\| \|\| \|\|\|\|\| \|\| \|\|\|\| \| \|\|\|\|\|\| \| \|\| \|\| \|\|\|\|\|\|\|\| | | |
| Sbjct: | 1233 | CGAGCGGGACGATGACCTGGAAACCTGTGGCTCCCCAGGGGGCCTCAAGGGTCGCTACTT | 1292 | |
| Query: | 849 | CTGGGCGGTGGGCGAGGAGGAGTTTGTCTGCGAGCCGCCCGTGGTGACTCACCGCTCACC | 908 | |
| | | \|\|\|\| \|\|\| \| \|\|\|\|\|\|\|\|\|\|\|\|\|\| \|\|\|\|\|\|\|\|\|\|\| \| \| \|\| \|\| \| \| \|\|\| | | |
| Sbjct: | 1293 | CTGGCATGTGCGTGAGGAGGAGTTTGTGTGCGAGCCGCCTCTCATCACCCAGCACACAC- | 1351 | |
| Query: | 909 | ACCTCTGGCTGTGCCCGCAGGTCG-GCCGGCTGCCCTGCGCTGCCGGGCAGTGGGGGACC | 967 | |
| | | \|\| \| \|\|\|\| \| \| \|\|\| \| \| \|\|\|\| \| \|\| \|\|\| \|\| \| \|\|\|\|\|\|\| | | |
| Sbjct: | 1352 | ACAAGTTGCTGGTTCTGGAGGGCCAGGCGGCCACACTCAAGTGCAAAGCCATTGGGGACC | 1411 | |
| Query: | 968 | CAGAGCCCCGTG-TGCGTTGGGTGTCACCCCAGGGCCGGCTGCTAGGCAACTCAAGCCGT | 1026 | |
| | | \| \|\|\|\|\|\| \| \| \| \|\|\|\|\| \| \|\|\| \| \| \|\|\| \|\|\| \|\|\|\| \|\|\|\|\| \| | | |
| Sbjct: | 1412 | CC-AGCCCCCTTATCCACTGGGTAGCCCCCGATGACCGCCTGGTAGGGAACTCCTCAAG- | 1469 | |
| Query: | 1027 | GCCCGC-GCCTTCCCCAATGGGACGCTGGAGCTGCTGGTCACCG-AGCCGGGTGATGGTG | 1084 | |
| | | \| \|\|\|\| \| \|\| \|\|\|\|\|\| \|\| \|\|\|\|\| \| \| \|\|\|\|\| \| \| \| \|\| \|\|\| | | |
| Sbjct: | 1470 | GACCGCTGTCTATGACAATGGCACCCTGGACATCTTCATCACCACATCTCAG-GACAGTG | 1528 | |
| Query: | 1085 | GCATCTTCACCTGCATTGCGGCCAATGCAGCTGGCGAGGCCACAGCTGCTGTGGAGCTGA | 1144 | |
| | | \| \|\|\|\|\|\|\|\|\|\|\|\|\|\|\| \|\|\|\|\|\|\|\| \|\| \|\| \|\|\|\|\|\|\|\| \|\| \|\|\|\|\|\| \| | | |
| Sbjct: | 1529 | GTGCCTTCACCTGCATTGCTGCCAATGCTGCCGGAGAGGCCACGGCCATGGTGGAGGTCT | 1588 | |
| Query: | 1145 | CTGTGGGTCCCCCA-CCACCTCCTCAGCTAGC-CAACAGCACCAGCTGTGACCCCCCGCG | 1202 | |
| | | \| \| \| \|\|\| \| \|\|\|\| \|\|\|\|\|\|\| \| \|\| \|\|\|\| \|\| \|\|\| \|\|\|\|\|\|\| \| | | |
| Sbjct: | 1589 | CCATCG-TCCAGCTGCCACA-CCTCAGCAACAGCACCAGCCGCA-CTGC-ACCCCCCAAG | 1644 | |
| Query: | 1203 | GGACGGGGATCCTG-ATGCTCTCACCCCACCCTCCGCTGCCTCTGCT-TCT-GCCAA-GG | 1258 | |
| | | \|\| \|\| \|\| \| \|\| \| \|\|\| \| \| \|\|\| \| \| \| \|\| \|\| | | |
| Sbjct: | 1645 | TCCCGCCTCTCAGACAT-CACTGGCTCCAGCAAGACCAGCCGGGGAGGTGGAGGCAGTGG | 1703 | |
| Query: | 1259 | TGGCCGACACTGGGCCC----CCTACCG-ACCGTGGC-GTCCAGGTGACTGAGCACGGGG | 1312 | |
| | | \|\|\| \|\| \| \|\| \|\| \|\|\| \| \|\| \|\|\| \|\| \| \|\|\| \|\|\|\| | | |
| Sbjct: | 1704 | GGGCGGAGAGCCTCCCAAAAGCCCCCCGGAACG-GGCTGTGCTTGTGTCTGAAGTGACCA | 1762 | |
| Query: | 1313 | CCACAGCTGCTCTTGTCCAGTGGCCGGATCAGCG-GCCT--ATCCCGGGCATCCGCATGT | 1369 | |
| | | \|\|\|\| \| \|\| \|\| \|\|\| \|\|\|\|\| \| \| \|\|\|\|\| \| \| \| \|\|\|\|\|\| \| \|\|\|\| | | |
| Sbjct: | 1763 | CCACCTCGGCCCTGGTCAAGTGGTCTG-TCAGCAAGTCAGCACCCCGGGTGAA-G-ATGT | 1819 | |
| Query: | 1370 | ACCAGATCCAGTACAACAGCTCGGCTGATGACATCCTCGTCTACAGGATGATCCCGGCGG | 1429 | |
| | | \|\|\|\|\| \| \|\|\|\|\|\|\|\|\| \|\|\|\| \| \|\|\|\|\| \| \|\| \| \|\|\|\|\|\|\|\|\|\|\|\|\|\| \|\| | | |
| Sbjct: | 1820 | ACCAGCTGCAGTACAACTGCTCTGACGATGAGGTACTGATTTACAGGATGATCCCAGCCT | 1879 | |
| Query: | 1430 | AGAGCCGCTCGTTCCTGCTGACGGACCTGGCGTCAGGC-CGGACCTACGATCTGTGCGTG | 1488 | |
| | | \| \| \| \|\|\| \|\| \| \| \|\|\|\|\|\| \|\|\|\|\|\| \| \| \| \|\|\|\|\| \|\|\|\| \|\|\| | | |
| Sbjct: | 1880 | CCAACAAGGCCTTCGTGGTCAACAACCTGGTGTCAGGGACTGGC-TACGACTTGTGTGTG | 1938 | |
| Query: | 1489 | CTCGCCGTGTATGAGGACAGCGCCACGGGGCTCACGGCCACGCGGCCT-GTGGGCTGCGC | 1547 | |
| | | \|\| \|\|\| \|\|\| \|\| \|\|\|\| \|\|\|\|\|\| \|\|\|\|\|\|\|\|\|\|\| \| \| \| \|\|\|\|\|\|\|\|\|\|\| | | |
| Sbjct: | 1939 | CTGGCCATGTGGGATGACACAGCCACGACACTCACGGCCAC-CAACATCGTGGGCTGCGC | 1997 | |
| Query: | 1548 | CCGCTTCTCCACCGAACCTGCGCTGCGGCCA-TGCGGGGCGCCGCAC-GCTCCCTTCCTG | 1605 | |
| | | \|\| \|\|\|\| \|\|\|\| \| \|\|\| \|\| \| \|\| \|\|\| \| \| \|\|\|\| \|\| \|\|\| \|\| | | |
| Sbjct: | 1998 | CCAGTTCTTCACCAAGGCTGA-CTACCCGCAGTGCCAGTCCATGCACAGCCAGATTC-TG | 2055 | |
| Query: | 1606 | GGCGGCACGATGATCATCGCGCTGGGCGGCGTCATCGTAGCCTCGGTACTGGTCTTCATC | 1665 | |
| | | \|\|\|\|\|\|\|\| \|\|\|\|\|\| \| \| \| \|\| \|\|\| \|\|\|\|\|\|\| \|\|\| \|\| \| \|\|\|\|\|\|\|\|\|\|\|\| | | |
| Sbjct: | 2056 | GGCGGCACCATGATCCTGGTCATCGGGGGCATCATCGTGGCCACGCTGCTGGTCTTCATC | 2115 | |
| Query: | 1666 | TTCGTGCTGCTAATGCGCTACAAGGTGCACGGCGGCCAGCCCCCCGGCAAGGCCAAGATT | 1725 | |
| | | \|\| \| \|\| \| \|\|\|\|\|\|\|\|\|\|\|\|\| \| \| \| \|\| \|\|\|\|\| \| \| \|\|\|\|\|\| | | |
| Sbjct: | 2116 | GTCATCCTCATGGTGCGCTACAAGGTCTGCAACCACGAGGCCCCC---A--G-CAAGATG | 2169 | |
| Query: | 1726 | CCCGCGCCTGTTAGCAGCGTTTGCTCCCAGACCAACGGCGCCCTGGGCCCCACGCCCACG | 1785 | |
| | | \| \|\|\| \| \|\| \|\|\|\| \|\| \| \|\|\| \|\|\|\|\|\|\|\|\|\|\|\|\|\|\|\| \| \| \|\| \| \|\|\| \| | | |
| Sbjct: | 2170 | GCAGCGGCCGTGAGCAATGTGTACTCGCAGACCAACGGCGCCCAGC-CACCGCCTCCAAG | 2228 | |
| Query: | 1786 | CCCGCCCCGCCCGCCCCGGAGCCCGCGGCGCTCAGGGCCCAC--ACCGTGGT | 1835 | |
| | | \| \|\| \| \|\| \|\|\| \|\| \|\|\|\| \|\| \|\|\| \|\|\|\|\|\|\| \| \| \|\|\|\|\| | | |
| Sbjct: | 2229 | CA-GCGCA-CCAGCCG-GG-GCCC-CGCCGC--AGGGCCCGCCGAAGGTGGT | 2273 | |

Alignment of TREMBLNEW-ACC:BAA86560 KIAA1246 PROTEIN - Homo sapiens
(Human), 832 aa (fragment) with a NOV2 polypeptide

BLASTX
>ptnr:TREMBLNEW-ACC:BAA86560 KIAA1246 PROTEIN -
Homo sapiens (Human), 832 aa (fragment).
Score = 1571 (553.0 bits), Expect = 1.0e-162, Sum P(2) = 1.0e-162
Identities = 313/581 (53%) , Positives = 402/581 (69%) , Frame = +1

| | | | | |
|---|---|---|---|---|
| Query: | 23 | ATPSPCPRRCRCQTQSLPLSVLCPGAGLLFVPPSLDRRAAELRLADNFIASVRRRDLANM | 82 | (SEQ ID NO:6) |
| | | \| \|\|+ \| \|\| \| \| \|\|\| \|\|\|\|\|\|\| +\|\|\| \|\|\|\| \|\|\| + \|+\| \|\|\| | | |
| Sbjct: | 59 | AVVDACPKYCVCQNLSESLGTLCPSKGLLFVPPDIDRRTVELRLGGNFIIHISRQDFANM | 118 | (SEQ ID NO:24) |
| Query: | 83 | TGLLHLSLSRNTIRHVAAGAFADLRALRALHLDGNRLTSLGEGQLRGLVNLRHLILSNNQ | 142 | |
| | | \|\|\|+ \|+\|\|\|\|\|\| \|+ +\| \|\| +\|\|+\|\|\|\| \|\|\| \|\|\|\| \|\|\|\|\|\|\|+\|\|\|++\|\|\| | | |
| Sbjct: | 119 | TGLVDLTLSRNTISHIQPFSFLDLESLRSLHLDSNRLPSLGEDTLRGLVNLQHLIVNNNQ | 178 | |
| Query: | 84 | LAALAAGALDDCAETLEDLDLSYNNLEQLPWEALGRLGNVNTLGLDHNLLASVPAGAFSR | 202 | |
| | | \| +\| \| +\| \|\|\|\|\|\|\|\|\|\|\|\| \|\|\|+++ \|+ \|++ \| \|\|\|\|\|\| + \| \|+ | | |
| Sbjct: | 179 | LGGIADEAFEDFLLTLEDLDLSYNNLHGLPWDSVRRMVNLHQLSLDHNLLDHIAEGTFAD | 238 | |
| Query: | 203 | LHKLARLDMTSNRLTTIPPDPLFSRLPLLARPRGSPASALVLAFGGNPLHCNCELVWLRR | 262 | |
| | | \| \|\|\|\|\|\|+\|\|\|\|\| +\|\|\|\|+\|+\| \| \| \| +\|\|\|\|\|\|\|\|\|\|\|\|+\|\|\|\| | | |
| Sbjct: | 239 | LQKLARLDLTSNRLQKLPPDPIFARSQASALTATPFAPPLSFSFGGNPLHCNCELLWLRR | 298 | |
| Query: | 263 | LAREDDLEACASPPALGGRYFWAVGEEEFVCEPPVVTHRSPPLAVPAGRPAALRCRAVGD | 322 | |
| | | \| \|+\|\|\|\| \| \|\| \| \|\|\|\|\| \| \|\|\|\|\|\|\|\|\|++\| + \| \| \|+ \| \|+\|+\|+\|\| | | |
| Sbjct: | 299 | LERDDDLETCGSPGGLKGRYFWHVREEEFVCEPPLITQHTHKLLVLEGQAATLKCKAIGD | 358 | |
| Query: | 323 | PEPRVRWVSPQGRLLGNSSRARAFPNGTLELLVTEPGDGGIFTCIAANAAGEATAAVELT | 382 | |
| | | \| \| + \|\|+\| \|\|+\|\|\|\|\| \| \|\|\|\|++ +\| \| \| \|\|\|\|\|\|\|\|\|\|\|\|\|\| \|\|++ | | |
| Sbjct: | 359 | PSPLIHWVAPDDRLVGNSSRTAVYDNGTLDIFITTSQDSGAFTCIAANAAGEATAMVEVS | 418 | |
| Query: | 383 | VGPPPPPQLANSTS-CDPPRDGDPDALTPPSAASASAKVADTG-PPT---DRGVQVTEHG | 437 | |
| | | + \| \|+\|\|\|\| \|\|+ \| +\| \| \| + \| \|\| +\| \| \|+\| | | |
| Sbjct: | 419 | I--VQLPHLSNSTSRTAPPKSRLSD-ITGSSKTSRGGGGSGGGEPPKSPPERAVLVSEVT | 475 | |
| Query: | 438 | ATAALVQWPDQRPIPGIRMYQIQYNSSADDILVYRMIPAESRSFLLTDLASGRTYDLCVL | 497 | |
| | | \|+\|\|\|+\| + \| ++\|\|\|+\|\|\| \| \|++\|+\|\|\|\|\|\| +++\|++ +\| \|\| \|\|\|\|\|\| | | |
| Sbjct: | 476 | TTSALVKWSVSKSAPRVKMYQLQYNCSDDEVLIYRMIPASNKAFVVNNLVSGTGYDLCVL | 535 | |
| Query: | 498 | AVYEDSATGLTATRPVGCARFSTEPALRPCGAPHAPFLGGTMIIALGGVIVASVLVFIFV | 557 | |
| | | \|+++\|+\|\| \|\|\|\| \|\|\|\|+\| \|+ \| + \|+ \|\|\|\|\|\|+ +\|\|+\|\|\|++\|\|\|\| + | | |
| Sbjct: | 536 | AMWDDTATTLTATNIVGCAQFFTKADYPQCQSMHSQILGGTMILVIGGIIVATLLVFIVI | 595 | |
| Query: | 558 | LLMRYKVHGGQPPGKAKIPAPVSSVCSQTNGALGPTPTPAPPAPEP | 603 | |
| | | \|++\|\|\|\| + \| +\|+ \| \|\|+\| \|\|\|\|\|\| \| \|+ \|\| \| | | |
| Sbjct: | 596 | LMVRYKVCNHEAP--SKMAAAVSNVYSQTNGAQPPPPSSAPAGAPP | 639 | |

This same alignment between KIAA1246 and NOV2 (identified as AF038458-A) is shown below in a ClustalW format. In this alignment, black outlined amino acid residues indicate regions of conserved sequence (i.e., regions that can be required to preserve structural or functional properties); greyed amino acid residues can be mutated to a residue with comparable steric and/or chemical properties without altering protein structure or function (ie L to V, I, or M); non-highlighted amino acid residues can potentially be mutated to a much broader extent without altering structure or function.

This same alignment between KIAA1246 and NOV2 (identified as AF038458A) is shown below.

[image]

Given that NOV2 polypeptides contain a signal peptide and appear to sort to a membrane, the NOV2 polypeptides are components of cell membranes. Carbohydrates, proteins and lipids are all important constituents of cell membranes. Proteins, including glycoproteins, which span the lipid bilayer are called transmembrane proteins and may cross back and forth several times. While hydrophobic residues are relegated to the middle of the membrane, this crossing back and forth exposes hydrophilic residues to the internal and external milieu. (Unwin, N. et al. (1984) Sci Am 250:78-87).

Membrane proteins participate in catalyzing reactions, transporting molecules in and out of cells, receiving and transmitting intercellular messages, anchoring cells to substratum, and providing tissue-identification tags. They are important in both paracrine and autocrine signaling and respond to growth factors. In fact, cells vary in identity, character, and bioactivity largely as a result of variation in their membrane proteins. Aberrant membrane proteins have been identified in a significant number of diseases and are intimately associated with cancers. Cells displaying these aberrant proteins are often locked in continuous cell division and are characterized by their altered gene transcription, decreased growth factor requirements, loss of anchorage, and cell morphology.

Based on it relatedness to transmembrane proteins that are expressed in brain, the NOV2 protein can be used to screen for ligands that are targeted to brain cells or that function as neurotransmitters. NOV2 genes and encoded proteins are useful in developing drugs for treating nervous disorders, and also for studying functions of the nervous system or onset mechanisms of nerve-related diseases. Also, the NOV2 polypeptide can identify brain cells from other types of tissue. The NOV2 polypeptide can also be used to identify signaling complexes that are linked to plasma membranes in brain cells. Nucleic acids, polypeptides, antibodies, and other compositions of the present invention will be useful in a variety of diseases and pathologies including the treatment of cancer, neurodegenerative disorders, Alzheimer's Disease, Parkinson's Disorder, immune disorders, and hematopoietic disorders.

NOV Nucleic Acids and Polypeptides

One aspect of the invention pertains to isolated nucleic acid molecules that encode NOV polypeptides or biologically-active portions thereof. Also included in the invention are nucleic acid fragments sufficient for use as hybridization probes to identify NOV-encoding nucleic acids (e.g., NOV mRNAs) and fragments for use as PCR primers for the amplification and/or mutation of NOV nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is comprised of double-stranded DNA.

A NOV nucleic acid can encode a mature NOV polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full length gene product, encoded by the corresponding gene. Alternatively, it can be defined as the polypeptide, precursor or proprotein encoded by an open reading frame described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes can be single- or double-stranded and designed to have specificity in PCR, membrane-based hybridization technologies, or ELISA-like technologies.

The term “isolated” nucleic acid molecule, as utilized herein, is one which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOV nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecules of the invention, e.g., a nucleic acid molecule having the nucleotide sequence of SEQ ID NO:1, 3, or 5, or a complement of this aforementioned nucleotide sequence, can be isolated using standard molecular biology techniques and the sequence information provided herein. Using all or a portion of the nucleic acid sequence of SEQ ID NO: 1, 3, or 5 as a hybridization probe, NOV molecules can be isolated using standard hybridization and cloning techniques (e.g., as described in Sambrook, et al., (eds.), MOLECULAR CLONING: A LABORATORY MANUAL 2nd Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acids of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOV nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence can be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO: 1, 3, or 5 or a complement thereof. Oligonucleotides can be chemically synthesized and may also be used as probes.

In another embodiment, an isolated nucleic acid molecule of the invention comprises a nucleic acid molecule that is a complement of the nucleotide sequence shown in SEQ ID NO: 1, 3, or 5, or a portion of this nucleotide sequence (e.g., a fragment that can be used as a probe or primer or a fragment encoding a biologically-active portion of a NOV polypeptide). A nucleic acid molecule that is complementary to the nucleotide sequence shown in SEQ ID NO: 1, 3, or 5, is one that is sufficiently complementary to the nucleotide sequence shown in SEQ ID NO: 1, 3, or 5, that it can hydrogen bond with little or no mismatches to the nucleotide sequence shown in SEQ ID NO: 1, 3, or 5, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions can be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments can be derived from any contiguous portion of a nucleic acid or amino acid sequence of choice.

Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs can be synthetic or from a different evolutionary origin and may have a similar or opposite metabolic activity compared to wild type. Homologs are nucleic acid sequences or amino acid sequences of a particular gene that are derived from different species.

Derivatives and analogs can be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 30%, 50%, 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOV polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOV polypeptide of species other than humans, including, but not limited to: vertebrates, and thus can include, e.g., frog, mouse, rat, rabbit, dog, cat, cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOV protein. Homologous nucleic acid sequences include those nucleic acid sequences that encode conservative amino acid substitutions (see below) in SEQ ID NO:2, 4, or 6, as well as a polypeptide possessing NOV biological activity. Various biological activities of the NOV proteins are described below.

A NOV polypeptide is encoded by the open reading frame (“ORF”) of a NOV nucleic acid. The invention includes the nucleic acid sequence comprising the stretch of nucleic acid sequences of SEQ ID NO: 1, 3, or 5, that comprises the ORF of that nucleic acid sequence and encodes a polypeptide of SEQ ID NO: 2, 4, or 6. The ORF can include the ORFs described above for the disclosed NOV sequences, e.g., the ORF beginning at nucleotides 5-7 and ending at nucleotides 467-469 for the disclosed NOV1a sequence (SEQ ID NO:1); the ORF beginning at nucleotides 5-7 and ending at nucleotides 602-604 for the disclosed NOV1b sequence (SEQ ID NO:3); and the ORF beginning at nucleotides 1-3 and ending at nucleotides 1885-1887 for the disclosed NOV2 sequence (SEQ ID NO:5).

An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF can be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the NOV genes allows for the generation of probes and primers designed for use in identifying and/or cloning NOV homologues in other cell types, e.g. from other tissues, as well as NOV homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:1, 3, or 5; or an anti-sense strand nucleotide sequence of SEQ ID NO:1, 3, or 5; or of a naturally occurring mutant of SEQ ID NO:1, 3, or 5.

Probes based on the NOV nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express a NOV protein, such as by measuring a level of a NOV-encoding nucleic acid in a sample of cells from a subject e.g., detecting NOV mRNA levels or determining whether a genomic NOV gene has been mutated or deleted.

“A polypeptide having a biologically-active portion of a NOV polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOV” can be prepared by isolating a portion of SEQ ID NO:1, 3, or 5, that encodes a polypeptide having a NOV biological activity (the biological activities of the NOV proteins are described below), expressing the encoded portion of NOV protein (e.g., by recombinant expression in vitro) and assessing the activity of the encoded portion of NOV.

NOV Nucleic Acid and Polypeptide Variants

The invention further encompasses nucleic acid molecules that differ from the nucleotide sequences shown in SEQ ID NO:1, 3, or 5, due to degeneracy of the genetic code and thus encode the same NOV proteins as that encoded by the nucleotide sequences shown in SEQ ID NO:1, 3, or 5. In another embodiment, an isolated nucleic acid molecule of the invention has a nucleotide sequence encoding a protein having an amino acid sequence shown in SEQ ID NO:2, 4, or 6.

In addition to the NOV nucleotide sequences shown in SEQ ID NO:1, 3, or 5, it will be appreciated by those skilled in the art that DNA sequence polymorphisms that lead to changes in the amino acid sequences of the NOV polypeptides may exist within a population (e.g., the human population). Such genetic polymorphism in the NOV genes may exist among individuals within a population due to natural allelic variation. As used herein, the terms “gene” and “recombinant gene” refer to nucleic acid molecules comprising an open reading frame (ORF) encoding a NOV protein, preferably a vertebrate NOV protein. Such natural allelic variations can typically result in 1-5% variance in the nucleotide sequence of the NOV genes. Any and all such nucleotide variations and resulting amino acid polymorphisms in the NOV polypeptides, which are the result of natural allelic variation and that do not alter the functional activity of the NOV polypeptides, are intended to be within the scope of the invention.

Moreover, nucleic acid molecules encoding NOV proteins from other species, and thus that have a nucleotide sequence that differs from the human sequence of SEQ ID NO:1, 3, or 5, are intended to be within the scope of the invention. Nucleic acid molecules corresponding to natural allelic variants and homologues of the NOV cDNAs of the invention can be isolated based on their homology to the human NOV nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:1, 3, or 5. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOV proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences of SEQ ID NO:1, 3, or 5, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that can hybridize to the nucleic acid molecule comprising the nucleotide sequence of SEQ ID NO:1, 3, 5 or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that can be used are well-known within the art. See, e.g., Ausubel ,et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that can hybridize to the nucleic acid molecule comprising the nucleotide sequences of SEQ ID NO:1, 3, 5 or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that can be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOV sequences that may exist in the population, the skilled artisan will further appreciate that changes can be introduced by mutation into the nucleotide sequences of SEQ ID NO:1, 3, or 5, thereby leading to changes in the amino acid sequences of the encoded NOV proteins, without altering the functional ability of said NOV proteins. For example, nucleotide substitutions leading to amino acid substitutions at “non-essential” amino acid residues can be made in the sequence of SEQ ID NO:2, 4, or 6. A “non-essential” amino acid residue is a residue that can be altered from the wild-type sequences of the NOV proteins without altering their biological activity, whereas an “essential” amino acid residue is required for such biological activity. For example, amino acid residues that are conserved among the NOV proteins of the invention are predicted to be particularly non-amenable to alteration. Amino acids for which conservative substitutions can be made are well-known within the art.

Another aspect of the invention pertains to nucleic acid molecules encoding NOV proteins that contain changes in amino acid residues that are not essential for activity. Such NOV proteins differ in amino acid sequence from SEQ ID NO:2, 4, or 6, yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NO:2, 4, or 6. Preferably, the protein encoded by the nucleic acid molecule is at least about 60% homologous to SEQ ID NO:2, 4, or 6; more preferably at least about 70% homologous to SEQ ID NO:2, 4, or 6; still more preferably at least about 80% homologous to SEQ ID NO:2, 4, or 6; even more preferably at least about 90% homologous to SEQ ID NO:2, 4, or 6; and most preferably at least about 95% homologous to SEQ ID NO:2, 4, or 6.

An isolated nucleic acid molecule encoding a NOV protein homologous to the protein of SEQ ID NO:2, 4, or 6, can be created by introducing one or more nucleotide substitutions, additions or deletions into the nucleotide sequence of SEQ ID NO:1, 3, or 5, such that one or more amino acid substitutions, additions or deletions are introduced into the encoded protein.

Mutations can be introduced into SEQ ID NO:2, 4, or 6, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOV protein is replaced with another amino acid residue from the same side chain family. Alternatively, in another embodiment, mutations can be introduced randomly along all or part of a NOV coding sequence, such as by saturation mutagenesis, and the resultant mutants can be screened for NOV biological activity to identify mutants that retain activity. Following mutagenesis of SEQ ID NO:2, 4, or 6, the encoded protein can be expressed by any recombinant technology known in the art and the activity of the protein can be determined.

In one embodiment, a mutant NOV protein can be assayed for (i) the ability to form protein:protein interactions with other NOV proteins, other cell-surface proteins, or biologically-active portions thereof, (ii) complex formation between a mutant NOV protein and a NOV ligand; or (iii) the ability of a mutant NOV protein to bind to an intracellular target protein or biologically-active portion thereof; (e.g. avidin proteins). In yet another embodiment, a mutant NOV protein can be assayed for the ability to regulate a specific biological function (e.g., regulation of insulin release).

Antisense Nucleic Acids

Another aspect of the invention pertains to isolated antisense nucleic acid molecules that can hybridize to or complementary to the nucleic acid molecule comprising the nucleotide sequence of SEQ ID NO:1, 3, 5 or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOV coding strand, or to only a portion thereof. Nucleic acid molecules encoding fragments, homologs, derivatives and analogs of a NOV protein of SEQ ID NO:2, 4, or 6; or antisense nucleic acids complementary to a NOV nucleic acid sequence of SEQ ID NO:1, 3, or 5, are additionally provided.

In one embodiment, an antisense nucleic acid molecule is antisense to a “coding region” of the coding strand of a nucleotide sequence encoding a NOV protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOV protein. The term “noncoding region” refers to 5′ and 3′ sequences which flank the coding region that are not translated into amino acids (i.e., also referred to as 5′ and 3′ untranslated regions).

Given the coding strand sequences encoding the NOV protein disclosed herein, antisense nucleic acids of the invention can be designed according tof the rules of Watson and Crick or Hoogsteen base pairing. The antisense nucleic acid molecule can be complementary to the entire coding region of NOV mRNA, but more preferably is an oligonucleotide that is antisense to only a portion of the coding or noncoding region of NOV mRNA. For example, the antisense oligonucleotide can be complementary to the region surrounding the translation start site of NOV mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOV protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an alpha-anomeric nucleic acid molecule. An alpha-anomeric nucleic acid molecule forms specific double-stranded hybrids with complementary RNA in which, contrary to the usual alpha-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they can be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOV mRNA transcripts to thereby inhibit translation of NOV mRNA. A ribozyme having specificity for a NOV-encoding nucleic acid can be designed based upon the nucleotide sequence of a NOV cDNA disclosed herein (i.e., SEQ ID NO:1, 3, or 5). For example, a derivative of a Tetrahymena L-19 IVS RNA can be constructed in which the nucleotide sequence of the active site is complementary to the nucleotide sequence to be cleaved in a NOV-encoding mRNA. See, e.g., U.S. Pat. No. 4,987,071 to Cech, et al. and U.S. Pat. No. 5,116,742 to Cech, et al. NOV mRNA can also be used to select a catalytic RNA having a specific ribonuclease activity from a pool of RNA molecules. See, e.g., Bartel et al., (1993) Science 261:1411-1418.

Alternatively, NOV gene expression can be inhibited by targeting nucleotide sequences complementary to the regulatory region of the NOV nucleic acid (e.g., the NOV promoter and/or enhancers) to form triple helical structures that prevent transcription of the NOV gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOV nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOV can be used in therapeutic and diagnostic applications. For example, PNAs can be used as antisense or antigene agents for sequence-specific modulation of gene expression by, e.g., inducing transcription or translation arrest or inhibiting replication. PNAs of NOV can also be used, for example, in the analysis of single base pair mutations in a gene (e.g., PNA directed PCR clamping; as artificial restriction enzymes when used in combination with other enzymes, e.g., S1 nucleases (see, Hyrup, et al., 1996.supra); or as probes or primers for DNA sequence and hybridization (see, Hyrup, et al., 1996, supra; Perry-O'Keefe, et al., 1996. supra).

In another embodiment, PNAs of NOV can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOV can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, etal., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide can be conjugated to another molecule, e.g., a peptide, a hybridization triggered cross-linking agent, a transport agent, a hybridization-triggered cleavage agent, and the like.

NOV Polypeptides

A polypeptide according to the invention includes a polypeptide including the amino acid sequence of NOV polypeptides whose sequences are provided in SEQ ID NO:2, 4, or 6. The invention also includes a mutant or variant protein any of whose residues can be changed from the corresponding residues shown in SEQ ID NO:2, 4, or 6, while still encoding a protein that maintains its NOV activities and physiological functions, or a functional fragment thereof.

In general, a NOV variant that preserves NOV-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOV proteins, and biologically-active portions thereof, or derivatives, fragments, analogs or homologs thereof. Also provided are polypeptide fragments suitable for use as immunogens to raise anti-NOV antibodies. In one embodiment, native NOV proteins can be isolated from cells or tissue sources by an appropriate purification scheme using standard protein purification techniques. In another embodiment, NOV proteins are produced by recombinant DNA techniques. Alternative to recombinant expression, a NOV protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOV protein is derived, or substantially free from chemical precursors or other chemicals when chemically synthesized. The language “substantially free of cellular material” includes preparations of NOV proteins in which the protein is separated from cellular components of the cells from which it is isolated or recombinantly-produced. In one embodiment, the language “substantially free of cellular material” includes preparations of NOV proteins having less than about 30% (by dry weight) of non-NOV proteins (also referred to herein as a “contaminating protein”), more preferably less than about 20% of non-NOV proteins, still more preferably less than about 10% of non-NOV proteins, and most preferably less than about 5% of non-NOV proteins. When the NOV protein or biologically-active portion thereof is recombinantly-produced, it is also preferably substantially free of culture medium, i.e., culture medium represents less than about 20%, more preferably less than about 10%, and most preferably less than about 5% of the volume of the NOV protein preparation.

The language “substantially free of chemical precursors or other chemicals” includes preparations of NOV proteins in which the protein is separated from chemical precursors or other chemicals that are involved in the synthesis of the protein. In one embodiment, the language “substantially free of chemical precursors or other chemicals” includes preparations of NOV proteins having less than about 30% (by dry weight) of chemical precursors or non-NOV chemicals, more preferably less than about 20% chemical precursors or non-NOV chemicals, still more preferably less than about 10% chemical precursors or non-NOV chemicals, and most preferably less than about 5% chemical precursors or non-NOV chemicals.

Biologically-active portions of NOV proteins include peptides comprising amino acid sequences sufficiently homologous to or derived from the amino acid sequences of the NOV proteins (e.g., the amino acid sequence shown in SEQ ID NO:2, 4, or 6) that include fewer amino acids than the full-length NOV proteins, and exhibit at least one activity of a NOV protein. Typically, biologically-active portions comprise a domain or motif with at least one activity of the NOV protein. A biologically-active portion of a NOV protein can be a polypeptide which is, for example, 10, 25, 50, 100 or more amino acid residues in length. Moreover, other biologically-active portions, in which other regions of the protein are deleted, can be prepared by recombinant techniques and evaluated for one or more of the functional activities of a native NOV protein.

In an embodiment, the NOV protein has an amino acid sequence shown in SEQ ID NO:2, 4, or 6. In other embodiments, the NOV protein is substantially homologous to SEQ ID NO:2, 4, or 6, and retains the functional activity of the protein of SEQ ID NO:2, 4, or 6, yet differs in amino acid sequence due to natural allelic variation or mutagenesis, as described in detail, below. Accordingly, in another embodiment, the NOV protein is a protein that comprises an amino acid sequence at least about 45% homologous to the amino acid sequence of SEQ ID NO:2, 4, or 6 and retains the functional activity of the NOV proteins of SEQ ID NO:2, 4, or 6.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology can be determined as the degree of identity between two sequences. The homology can be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO:1, 3, or 5.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOV chimeric or fusion proteins. As used herein, a NOV “chimeric protein” or “fusion protein” comprises a NOV polypeptide operatively-linked to a non-NOV polypeptide. An “NOV polypeptide” refers to a polypeptide having an amino acid sequence corresponding to a NOV protein (SEQ ID NO:2, 4, or 6), whereas a “non-NOV polypeptide” refers to a polypeptide having an amino acid sequence corresponding to a protein that is not substantially homologous to the NOV protein, e.g., a protein that is different from the NOV protein and that is derived from the same or a different organism. Within a NOV fusion protein the NOV polypeptide can correspond to all or a portion of a NOV protein. In one embodiment, a NOV fusion protein comprises at least one biologically-active portion of a NOV protein. In another embodiment, a NOV fusion protein comprises at least two biologically-active portions of a NOV protein. In yet another embodiment, a NOV fusion protein comprises at least three biologically-active portions of a NOV protein. Within the fusion protein, the term “operatively-linked” is intended to indicate that the NOV polypeptide and the non-NOV polypeptide are fused in-frame with one another. The non-NOV polypeptide can be fused to the N-terminus or C-terminus of the NOV polypeptide.

In one embodiment, the fusion protein is a GST-NOV fusion protein in which the NOV sequences are fused to the C-terminus of the GST (glutathione S-transferase) sequences. Such fusion proteins can facilitate the purification of recombinant NOV polypeptides.

In another embodiment, the fusion protein is a NOV protein containing a heterologous signal sequence at its N-terminus. In certain host cells (e.g., mammalian host cells), expression and/or secretion of NOV can be increased through use of a heterologous signal sequence.

In yet another embodiment, the fusion protein is a NOV-immunoglobulin fusion protein in which the NOV sequences are fused to sequences derived from a member of the immunoglobulin protein family. The NOV-immunoglobulin fusion proteins of the invention can be incorporated into pharmaceutical compositions and administered to a subject to inhibit an interaction between a NOV ligand and a NOV protein on the surface of a cell, to thereby suppress NOV-mediated signal transduction in vivo. The NOV-immunoglobulin fusion proteins can be used to affect the bioavailability of a NOV cognate ligand. Inhibition of the NOV ligand/NOV interaction can be useful therapeutically for both the treatment of proliferative and differentiative disorders, as well as modulating (e.g. promoting or inhibiting) cell survival. Moreover, the NOV-immunoglobulin fusion proteins of the invention can be used as immunogens to produce anti-NOV antibodies in a subject, to purify NOV ligands, and in screening assays to identify molecules that inhibit the interaction of NOV with a NOV ligand.

A NOV chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOV-encoding nucleic acid can be cloned into such an expression vector such that the fusion moiety is linked in-frame to the NOV protein.

NOV Agonists and Antagonists

The invention also pertains to variants of the NOV proteins that function as either NOV agonists (i.e., mimetics) or as NOV antagonists. Variants of the NOV protein can be generated by mutagenesis (e.g., discrete point mutation or truncation of the NOV protein). An agonist of the NOV protein can retain substantially the same, or a subset of, the biological activities of the naturally occurring form of the NOV protein. An antagonist of the NOV protein can inhibit one or more of the activities of the naturally occurring form of the NOV protein by, for example, competitively binding to a downstream or upstream member of a cellular signaling cascade which includes the NOV protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOV proteins.

Variants of the NOV proteins that function as either NOV agonists (i.e., mimetics) or as NOV antagonists can be identified by screening combinatorial libraries of mutants (e.g., truncation mutants) of the NOV proteins for NOV protein agonist or antagonist activity. In one embodiment, a variegated library of NOV variants is generated by combinatorial mutagenesis at the nucleic acid level and is encoded by a variegated gene library. A variegated library of NOV variants can be produced by, for example, enzymatically ligating a mixture of synthetic oligonucleotides into gene sequences such that a degenerate set of potential NOV sequences is expressible as individual polypeptides, or alternatively, as a set of larger fusion proteins (e.g., for phage display) containing the set of NOV sequences therein. There are a variety of methods which can be used to produce libraries of potential NOV variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOV sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11:477.

Polypeptide Libraries

In addition, libraries of fragments of the NOV protein coding sequences can be used to generate a variegated population of NOV fragments for screening and subsequent selection of variants of a NOV protein. In one embodiment, a library of coding sequence fragments can be generated by treating a double stranded PCR fragment of a NOV coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S1 nuclease, and ligating the resulting fragment library into an expression vector. By this method, expression libraries can be derived which encodes N-terminal and internal fragments of various sizes of the NOV proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOV proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOV variants. See, e.g., Arkin and Yourvan, 1992. Proc. Natl. Acad. Sci. USA 89: 7811-7815; Delgrave, et al., 1993. Protein Engineering 6:327-331.

Anti-NOV Antibodies

The invention encompasses antibodies and antibody fragments, such as Fab or (Fab)₂, that bind immunospecifically to any of the NOV polypeptides of said invention.

An isolated NOV protein, or a portion or fragment thereof, can be used as an immunogen to generate antibodies that bind to NOV polypeptides using standard techniques for polyclonal and monoclonal antibody preparation. The full-length NOV proteins can be used or, alternatively, the invention provides antigenic peptide fragments of NOV proteins for use as immunogens. The antigenic NOV peptides comprises at least 4 amino acid residues of the amino acid sequence shown in SEQ ID NO:2, 4, or 6, and encompasses an epitope of NOV such that an antibody raised against the peptide forms a specific immune complex with NOV. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of NOV that is located on the surface of the protein (e.g., a hydrophilic region). As a means for targeting antibody production, hydropathy plots showing regions of hydrophilicity and hydrophobicity can be generated by any method well known in the art, including, for example, the Kyte Doolittle or the Hopp Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

As disclosed herein, NOV protein sequences of SEQ ID NO:2, 4, 6 or derivatives, fragments, analogs or homologs thereof, can be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as NOV. Such antibodies include, but are not limited to, polyclonal, monoclonal, chimeric, single chain, Fab and F(ab′)₂ fragments, and an Fab expression library. In a specific embodiment, antibodies to human NOV proteins are disclosed. Various procedures known within the art can be used for the production of polyclonal or monoclonal antibodies to a NOV protein sequence of SEQ ID NO:2, 4, 6 or a derivative, fragment, analog or homolog thereof. Some of these proteins are discussed below.

For the production of polyclonal antibodies, various suitable host animals (e.g., rabbit, goat, mouse or other mammal) can be immunized by injection with the native protein, or a synthetic variant thereof, or a derivative of the foregoing. An appropriate immunogenic preparation can contain, for example, recombinantly-expressed NOV protein or a chemically-synthesized NOV polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against NOV can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of NOV. A monoclonal antibody composition thus typically displays a single binding affinity for a particular NOV protein with which it immunoreacts. For preparation of monoclonal antibodies directed towards a particular NOV protein, or derivatives, fragments, analogs or homologs thereof, any technique that provides for the production of antibody molecules by continuous cell line culture can be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies can be utilized in the practice of the invention and can be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to a NOV protein (see, e.g., U.S. Pat. No. 4,946,778). In addition, methods can be adapted for the construction of Fab expression libraries (see, e.g., Huse, et al., 1989. Science 246: 1275-1281) to allow rapid and effective identification of monoclonal Fab fragments with the desired specificity for a NOV protein or derivatives, fragments, analogs or homologs thereof. Non-human antibodies can be “humanized” by techniques well known in the art. See, e.g., U.S. Pat. No. 5,225,539. Antibody fragments that contain the idiotypes to a NOV protein can be produced by techniques known in the art including, but not limited to: (i) an F(ab′)₂ fragment produced by pepsin digestion of an antibody molecule; (ii) an Fab fragment generated by reducing the disulfide bridges of an F(ab′)₂ fragment; (iii) an Fab fragment generated by the treatment of the antibody molecule with papain and a reducing agent; and (iv) Fv fragments.

Additionally, recombinant anti-NOV antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. Nos. 4,816,567; 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314:446-449; Shaw, et al., 1988. J. Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a NOV protein is facilitated by generation of hybridomas that bind to the fragment of a NOV protein possessing such a domain. Thus, antibodies that are specific for a desired domain within a NOV protein, or derivatives, fragments, analogs or homologs thereof, are also provided herein.

Anti-NOV antibodies can be used in methods known within the art relating to the localization and/or quantitation of a NOV protein (e.g., for use in measuring levels of the NOV protein within appropriate physiological samples, for use in diagnostic methods, for use in imaging the protein, and the like). In a given embodiment, antibodies for NOV proteins, or derivatives, fragments, analogs or homologs thereof, that contain the antibody derived binding domain, are utilized as pharmacologically-active compounds (hereinafter “Therapeutics”).

An anti-NOV antibody (e.g., monoclonal antibody) can be used to isolate a NOV polypeptide by standard techniques, such as affinity chromatography or immunoprecipitation. An anti-NOV antibody can facilitate the purification of natural NOV polypeptide from cells and of recombinantly-produced NOV polypeptide expressed in host cells. Moreover, an anti-NOV antibody can be used to detect NOV protein (e.g., in a cellular lysate or cell supernatant) in order to evaluate the abundance and pattern of expression of the NOV protein. Anti-NOV antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOV Recombinant Expression Vectors and Host Cells

Another aspect of the invention pertains to vectors, preferably expression vectors, containing a nucleic acid encoding a NOV protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOV proteins, mutant forms of NOV proteins, fusion proteins, etc.).

The recombinant expression vectors of the invention can be designed for expression of NOV proteins in prokaryotic or eukaryotic cells. For example, NOV proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOV expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). Alternatively, NOV can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the cc-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOV mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOV protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOV or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOV protein. Accordingly, the invention further provides methods for producing NOV protein using the host cells of the invention. In one embodiment, the method comprises culturing the host cell of invention (into which a recombinant expression vector encoding NOV protein has been introduced) in a suitable medium such that NOV protein is produced. In another embodiment, the method further comprises isolating NOV protein from the medium or the host cell.

Transgenic NOV Animals

The host cells of the invention can also be used to produce non-human transgenic animals. For example, in one embodiment, a host cell of the invention is a fertilized oocyte or an embryonic stem cell into which NOV protein-coding sequences have been introduced. Such host cells can then be used to create non-human transgenic animals in which exogenous NOV sequences have been introduced into their genome or homologous recombinant animals in which endogenous NOV sequences have been altered. Such animals are useful for studying the function and/or activity of NOV protein and for identifying and/or evaluating modulators of NOV protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOV gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOV-encoding nucleic acid into the male pronuclei of a fertilized oocyte (e.g., by microinjection, retroviral infection) and allowing the oocyte to develop in a pseudopregnant female foster animal. The human NOV cDNA sequences of SEQ ID NO:1, 3, or 5, can be introduced as a transgene into the genome of a non-human animal. Alternatively, a non-human homologue of the human NOV gene, such as a mouse NOV gene, can be isolated based on hybridization to the human NOV cDNA (described further supra) and used as a transgene. Intronic sequences and polyadenylation signals can also be included in the transgene to increase the efficiency of expression of the transgene. A tissue-specific regulatory sequence(s) can be operably-linked to the NOV transgene to direct expression of NOV protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOV transgene in its genome and/or expression of NOV mRNA in tissues or cells of the animals. A transgenic founder animal can then be used to breed additional animals carrying the transgene. Moreover, transgenic animals carrying a transgene-encoding NOV protein can further be bred to other transgenic animals carrying other transgenes.

To create a homologous recombinant animal, a vector is prepared which contains at least a portion of a NOV gene into which a deletion, addition or substitution has been introduced to thereby alter, e.g., functionally disrupt, the NOV gene. The NOV gene can be a human gene (e.g., the cDNA of SEQ ID NO:1, 3, or 5), but more preferably, is a non-human homologue of a human NOV gene. For example, a mouse homologue of human NOV gene of SEQ ID NO:1, 3, or 5, can be used to construct a homologous recombination vector suitable for altering an endogenous NOV gene in the mouse genome. In one embodiment, the vector is designed such that, upon homologous recombination, the endogenous NOV gene is functionally disrupted (i.e., no longer encodes a functional protein; also referred to as a “knock out” vector).

Alternatively, the vector can be designed such that, upon homologous recombination, the endogenous NOV gene is mutated or otherwise altered but still encodes functional protein (e.g., the upstream regulatory region can be altered to thereby alter the expression of the endogenous NOV protein). In the homologous recombination vector, the altered portion of the NOV gene is flanked at its 5′- and 3′-termini by additional nucleic acid of the NOV gene to allow for homologous recombination to occur between the exogenous NOV gene carried by the vector and an endogenous NOV gene in an embryonic stem cell. The additional flanking NOV nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is then introduced into an embryonic stem cell line (e.g., by electroporation) and cells in which the introduced NOV gene has homologously-recombined with the endogenous NOV gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G0 phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOV nucleic acid molecules, NOV proteins, and anti-NOV antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOV protein or anti-NOV antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

Screening and Detection Methods

The isolated nucleic acid molecules of the invention can be used to express NOV protein (e.g., via a recombinant expression vector in a host cell in gene therapy applications), to detect NOV mRNA (e.g., in a biological sample) or a genetic lesion in a NOV gene, and to modulate NOV activity, as described further, below. In addition, the NOV proteins can be used to screen drugs or compounds that modulate the NOV protein activity or expression as well as to treat disorders characterized by insufficient or excessive production of NOV protein or production of NOV protein forms that have decreased or aberrant activity compared to NOV wild-type protein. In addition, the anti-NOV antibodies of the invention can be used to detect and isolate NOV proteins and modulate NOV activity.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOV proteins or have a stimulatory or inhibitory effect on, e.g., NOV protein expression or NOV protein activity. The invention also includes compounds identified in the screening assays described herein.

In one embodiment, the invention provides assays for screening candidate or test compounds which bind to or modulate the activity of the membrane-bound form of a NOV protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds can be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOV protein, or a biologically-active portion thereof, on the cell surface is contacted with a test compound and the ability of the test compound to bind to a NOV protein determined. The cell, for example, can of mammalian origin or a yeast cell. Determining the ability of the test compound to bind to the NOV protein can be accomplished, for example, by coupling the test compound with a radioisotope or enzymatic label such that binding of the test compound to the NOV protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOV protein, or a biologically-active portion thereof, on the cell surface with a known compound which binds NOV to form an assay mixture, contacting the assay mixture with a test compound, and determining the ability of the test compound to interact with a NOV protein, wherein determining the ability of the test compound to interact with a NOV protein comprises determining the ability of the test compound to preferentially bind to NOV protein or a biologically-active portion thereof as compared to the known compound.

In another embodiment, an assay is a cell-based assay comprising contacting a cell expressing a membrane-bound form of NOV protein, or a biologically-active portion thereof, on the cell surface with a test compound and determining the ability of the test compound to modulate (e.g., stimulate or inhibit) the activity of the NOV protein or biologically-active portion thereof.

Determining the ability of the test compound to modulate the activity of NOV or a biologically-active portion thereof can be accomplished, for example, by determining the ability of the NOV protein to bind to or interact with a NOV target molecule. As used herein, a “target molecule” is a molecule with which a NOV protein binds or interacts in nature, for example, a molecule on the surface of a cell which expresses a NOV interacting protein, a molecule on the surface of a second cell, a molecule in the extracellular milieu, a molecule associated with the internal surface of a cell membrane or a cytoplasmic molecule. A NOV target molecule can be a non-NOV molecule or a NOV protein or polypeptide of the invention. In one embodiment, a NOV target molecule is a component of a signal transduction pathway that facilitates transduction of an extracellular signal (e.g. a signal generated by binding of a compound to a membrane-bound NOV molecule) through the cell membrane and into the cell. The target, for example, can be a second intercellular protein that has catalytic activity or a protein that facilitates the association of downstream signaling molecules with NOV.

Determining the ability of the NOV protein to bind to or interact with a NOV target molecule can be accomplished by one of the methods described above for determining direct binding. In one embodiment, determining the ability of the NOV protein to bind to or interact with a NOV target molecule can be accomplished by determining the activity of the target molecule. For example, the activity of the target molecule can be determined by detecting induction of a cellular second messenger of the target (i.e. intracellular Ca²⁺, diacylglycerol, IP3, etc.), detecting catalytic/enzymatic activity of the target an appropriate substrate, detecting the induction of a reporter gene (comprising a NOV-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOV protein or biologically-active portion thereof with a test compound and determining the ability of the test compound to bind to the NOV protein or biologically-active portion thereof. Binding of the test compound to the NOV protein can be determined either directly or indirectly as described above. In one such embodiment, the assay comprises contacting the NOV protein or biologically-active portion thereof with a known compound which binds NOV to form an assay mixture, contacting the assay mixture with a test compound, and determining the ability of the test compound to interact with a NOV protein, wherein determining the ability of the test compound to interact with a NOV protein comprises determining the ability of the test compound to preferentially bind to NOV or biologically-active portion thereof as compared to the known compound.

In still another embodiment, an assay is a cell-free assay comprising contacting NOV protein or biologically-active portion thereof with a test compound and determining the ability of the test compound to modulate (e.g. stimulate or inhibit) the activity of the NOV protein or biologically-active portion thereof. Determining the ability of the test compound to modulate the activity of NOV can be accomplished, for example, by determining the ability of the NOV protein to bind to a NOV target molecule by one of the methods described above for determining direct binding. In an alternative embodiment, determining the ability of the test compound to modulate the activity of NOV protein can be accomplished by determining the ability of the NOV protein to further modulate a NOV target molecule. For example, the catalytic/enzymatic activity of the target molecule on an appropriate substrate can be determined as described, supra.

In yet another embodiment, the cell-free assay comprises contacting the NOV protein or biologically-active portion thereof with a known compound which binds NOV protein to form an assay mixture, contacting the assay mixture with a test compound, and determining the ability of the test compound to interact with a NOV protein, wherein determining the ability of the test compound to interact with a NOV protein comprises determining the ability of the NOV protein to preferentially bind to or modulate the activity of a NOV target molecule.

The cell-free assays of the invention are amenable to use of both the soluble form or the membrane-bound form of NOV protein. In the case of cell-free assays comprising the membrane-bound form of NOV protein, it can be desirable to utilize a solubilizing agent such that the membrane-bound form of NOV protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)n, N-dodecyl--N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it can be desirable to immobilize either NOV protein or its target molecule to facilitate separation of complexed from uncomplexed forms of one or both of the proteins, as well as to accommodate automation of the assay. Binding of a test compound to NOV protein, or interaction of NOV protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOV fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOV protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOV protein binding or activity determined using standard techniques.

Other techniques for immobilizing proteins on matrices can also be used in the screening assays of the invention. For example, either the NOV protein or its target molecule can be immobilized utilizing conjugation of biotin and streptavidin. Biotinylated NOV protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOV protein or target molecules, but which do not interfere with binding of the NOV protein to its target molecule, can be derivatized to the wells of the plate, and unbound target or NOV protein trapped in the wells by antibody conjugation. Methods for detecting such complexes, in addition to those described above for the GST-immobilized complexes, include immunodetection of complexes using antibodies reactive with the NOV protein or target molecule, as well as enzyme-linked assays that rely on detecting an enzymatic activity associated with the NOV protein or target molecule.

In another embodiment, modulators of NOV protein expression are identified in a method wherein a cell is contacted with a candidate compound and the expression of NOV mRNA or protein in the cell is determined. The level of expression of NOV mRNA or protein in the presence of the candidate compound is compared to the level of expression of NOV mRNA or protein in the absence of the candidate compound. The candidate compound can then be identified as a modulator of NOV mRNA or protein expression based upon this comparison. For example, when expression of NOV mRNA or protein is greater (i.e., statistically significantly greater) in the presence of the candidate compound than in its absence, the candidate compound is identified as a stimulator of NOV mRNA or protein expression. Alternatively, when expression of NOV mRNA or protein is less (statistically significantly less) in the presence of the candidate compound than in its absence, the candidate compound is identified as an inhibitor of NOV mRNA or protein expression. The level of NOV mRNA or protein expression in the cells can be determined by methods described herein for detecting NOV mRNA or protein.

In yet another aspect of the invention, the NOV proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOV (“NOV-binding proteins” or “NOV-bp”) and modulate NOV activity. Such NOV-binding proteins are also likely to be involved in the propagation of signals by the NOV proteins as, for example, upstream or downstream elements of the NOV pathway.

The two-hybrid system is based on the modular nature of most transcription factors, which consist of separable DNA-binding and activation domains. Briefly, the assay utilizes two different DNA constructs. In one construct, the gene that codes for NOV is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOV-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOV.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOV sequences, SEQ ID NO:1, 3, or 5, or fragments or derivatives thereof, can be used to map the location of the NOV genes, respectively, on a chromosome. The mapping of the NOV sequences to chromosomes is an important first step in correlating these sequences with genes associated with disease.

Briefly, NOV genes can be mapped to chromosomes by preparing PCR primers (preferably 15-25 bp in length) from the NOV sequences. Computer analysis of the NOV, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOV sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOV sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOV gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOV sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOV sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOV sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NO:1, 3, or 5, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOV protein and/or nucleic acid expression as well as NOV activity, in the context of a biological sample (e.g., blood, serum, cells, tissue) to thereby determine whether an individual is afflicted with a disease or disorder, or is at risk of developing a disorder, associated with aberrant NOV expression or activity.

NOV1 is a member of the interferon family. Interferons are part of the body's natural defense to viruses and tumors. Accordingly, diseases associated with aberrant NOV1 expression include viral infections, cancer, neurologic diseases, and acute lymphoblastic leukemia and gliomas. NOV2 is a member of the transmembrane protein family. Aberrant membrane proteins have been identified in a significant number of diseases, including cancer, neurodegenerative disorders, Alzheimer's Disease, Parkinson's Disorder, immune disorders, and hematopoietic disorders.

The invention also provides for prognostic (or predictive) assays for determining whether an individual is at risk of developing a disorder associated with NOV protein, nucleic acid expression or activity. For example, mutations in a NOV gene can be assayed in a biological sample. Such assays can be used for prognostic or predictive purpose to thereby prophylactically treat an individual prior to the onset of a disorder characterized by or associated with NOV protein, nucleic acid expression, or biological activity.

Another aspect of the invention provides methods for determining NOV protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOV in clinical trials.

These and other agents are described in further detail in the following sections.

Diagnostic Assays

An exemplary method for detecting the presence or absence of NOV in a biological sample involves obtaining a biological sample from a test subject and contacting the biological sample with a compound or an agent capable of detecting NOV protein or nucleic acid (e.g., mRNA, genomic DNA) that encodes NOV protein such that the presence of NOV is detected in the biological sample. An agent for detecting NOV mRNA or genomic DNA is a labeled nucleic acid probe capable of hybridizing to NOV mRNA or genomic DNA. The nucleic acid probe can be, for example, a full-length NOV nucleic acid, such as the nucleic acid of SEQ ID NO:1, 3, 5 or a portion thereof, such as an oligonucleotide of at least 15, 30, 50, 100, 250 or 500 nucleotides in length and sufficient to specifically hybridize under stringent conditions to NOV mRNA or genomic DNA. Other suitable probes for use in the diagnostic assays of the invention are described herein.

An agent for detecting NOV protein is an antibody capable of binding to NOV protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOV mRNA, protein, or genomic DNA in a biological sample in vitro as well as in vivo. For example, in vitro techniques for detection of NOV mRNA include Northern hybridizations and in situ hybridizations. In vitro techniques for detection of NOV protein include enzyme linked immunosorbent assays (ELISAs), Western blots, immunoprecipitations, and immunofluorescence. In vitro techniques for detection of NOV genomic DNA include Southern hybridizations. Furthermore, in vivo techniques for detection of NOV protein include introducing into a subject a labeled anti-NOV antibody. For example, the antibody can be labeled with a radioactive marker whose presence and location in a subject can be detected by standard imaging techniques. In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOV protein, mRNA, or genomic DNA, such that the presence of NOV protein, mRNA or genomic DNA is detected in the biological sample, and comparing the presence of NOV protein, mRNA or genomic DNA in the control sample with the presence of NOV protein, mRNA or genomic DNA in the test sample.

The invention also encompasses kits for detecting the presence of NOV in a biological sample. For example, the kit can comprise: a labeled compound or agent capable of detecting NOV protein or mRNA in a biological sample; means for determining the amount of NOV in the sample; and means for comparing the amount of NOV in the sample with a standard. The compound or agent can be packaged in a suitable container. The kit can further comprise instructions for using the kit to detect NOV protein or nucleic acid.

Prognostic Assays

The diagnostic methods described herein can furthermore be utilized to identify subjects having or at risk of developing a disease or disorder associated with aberrant NOV expression or activity.

NOV1 is a member of the interferon family. Interferons are part of the body's natural defense to viruses and tumors. Accordingly, diseases associated with aberrant NOV1 expression include viral infections, cancer, neurologic diseases, and acute lymphoblastic leukemia and gliomas. NOV2 is a member of the transmembrane protein family. Aberrant membrane proteins have been identified in a significant number of diseases, including cancer, neurodegenerative disorders, Alzheimer's Disease, Parkinson's Disorder, immune disorders, and hematopoietic disorders.

For example, the assays described herein, such as the preceding diagnostic assays or the following assays, can be utilized to identify a subject having or at risk of developing a disorder associated with NOV protein, nucleic acid expression or activity. Alternatively, the prognostic assays can be utilized to identify a subject having or at risk for developing a disease or disorder. Thus, the invention provides a method for identifying a disease or disorder associated with aberrant NOV expression or activity in which a test sample is obtained from a subject and NOV protein or nucleic acid (e.g., mRNA, genomic DNA) is detected, wherein the presence of NOV protein or nucleic acid is diagnostic for a subject having or at risk of developing a disease or disorder associated with aberrant NOV expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOV expression or activity. For example, such methods can be used to determine whether a subject can be effectively treated with an agent for a disorder. Thus, the invention provides methods for determining whether a subject can be effectively treated with an agent for a disorder associated with aberrant NOV expression or activity in which a test sample is obtained and NOV protein or nucleic acid is detected (e.g., wherein the presence of NOV protein or nucleic acid is diagnostic for a subject that can be administered the agent to treat a disorder associated with aberrant NOV expression or activity).

The methods of the invention can also be used to detect genetic lesions in a NOV gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOV-protein, or the misexpression of the NOV gene. For example, such genetic lesions can be detected by ascertaining the existence of at least one of: (i) a deletion of one or more nucleotides from a NOV gene; (ii) an addition of one or more nucleotides to a NOV gene; (iii) a substitution of one or more nucleotides of a NOV gene, (iv) a chromosomal rearrangement of a NOV gene; (v) an alteration in the level of a messenger RNA transcript of a NOV gene, (vi) aberrant modification of a NOV gene, such as of the methylation pattern of the genomic DNA, (vii) the presence of a non-wild-type splicing pattern of a messenger RNA transcript of a NOV gene, (viii) a non-wild-type level of a NOV protein, (ix) allelic loss of a NOV gene, and (x) inappropriate post-translational modification of a NOV protein. As described herein, there are a large number of assay techniques known in the art which can be used for detecting lesions in a NOV gene. A preferred biological sample is a peripheral blood leukocyte sample isolated by conventional means from a subject. However, any biological sample containing nucleated cells can be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOV-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOV gene under conditions such that hybridization and amplification of the NOV gene (if present) occurs, and detecting the presence or absence of an amplification product, or detecting the size of the amplification product and comparing the length to a control sample. It is anticipated that PCR and/or LCR can be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Q Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOV gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOV can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOV can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOV gene and detect mutations by comparing the sequence of the sample NOV with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOV gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOV sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOV cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOV sequence, e.g., a wild-type NOV sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOV genes. For example, single strand conformation polymorphism (SSCP) can be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOV nucleic acids will be denatured and allowed to renature. The secondary structure of single-stranded nucleic acids varies according to sequence, the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers can be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification can be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it can be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein can be performed, for example, by utilizing pre-packaged diagnostic kits comprising at least one probe nucleic acid or antibody reagent described herein, which can be conveniently used, e.g., in clinical settings to diagnose patients exhibiting symptoms or family history of a disease or illness involving a NOV gene. Furthermore, any cell type or tissue, preferably peripheral blood leukocytes, in which NOV is expressed can be utilized in the prognostic assays described herein. However, any biological sample containing nucleated cells can be used, including, for example, buccal mucosal cells.

Pharmacogenomics

Agents, or modulators that have a stimulatory or inhibitory effect on NOV activity (e.g., NOV gene expression), as identified by a screening assay described herein can be administered to individuals to treat (prophylactically or therapeutically) disorders (e.g., cancer or immune disorders associated with aberrant NOV activity. In conjunction with such treatment, the pharmacogenomics (i.e., the study of the relationship between an individual's genotype and that individual's response to a foreign compound or drug) of the individual can be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOV protein, expression of NOV nucleic acid, or mutation content of NOV genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOV protein, expression of NOV nucleic acid, or mutation content of NOV genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOV modulator, such as a modulator identified by one of the exemplary screening assays described herein.

Monitoring of Effects During Clinical Trials

Monitoring the influence of agents (e.g., drugs, compounds) on the expression or activity of NOV (e.g., the ability to modulate aberrant cell proliferation and/or differentiation) can be applied not only in basic drug screening, but also in clinical trials. For example, the effectiveness of an agent determined by a screening assay as described herein to increase NOV gene expression, protein levels, or upregulate NOV activity, can be monitored in clinical trails of subjects exhibiting decreased NOV gene expression, protein levels, or downregulated NOV activity. Alternatively, the effectiveness of an agent determined by a screening assay to decrease NOV gene expression, protein levels, or downregulate NOV activity, can be monitored in clinical trails of subjects exhibiting increased NOV gene expression, protein levels, or upregulated NOV activity. In such clinical trials, the expression or activity of NOV and, preferably, other genes that have been implicated in, for example, a cellular proliferation or immune disorder can be used as a “read out” or markers of the immune responsiveness of a particular cell.

By way of example, and not of limitation, genes, including NOV, that are modulated in cells by treatment with an agent (e.g., compound, drug or small molecule) that modulates NOV activity (e.g., identified in a screening assay as described herein) can be identified. Thus, to study the effect of agents on cellular proliferation disorders, for example, in a clinical trial, cells can be isolated and RNA prepared and analyzed for the levels of expression of NOV and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOV or other genes. In this manner, the gene expression pattern can serve as a marker, indicative of the physiological response of the cells to the agent. Accordingly, this response state can be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOV protein, mRNA, or genomic DNA in the preadministration sample; (iii) obtaining one or more post-administration samples from the subject; (iv) detecting the level of expression or activity of the NOV protein, mRNA, or genomic DNA in the post-administration samples; (v) comparing the level of expression or activity of the NOV protein, mRNA, or genomic DNA in the pre-administration sample with the NOV protein, mRNA, or genomic DNA in the post administration sample or samples; and (vi) altering the administration of the agent to the subject accordingly. For example, increased administration of the agent can be desirable to increase the expression or activity of NOV to higher levels than detected, i.e., to increase the effectiveness of the agent. Alternatively, decreased administration of the agent can be desirable to decrease expression or activity of NOV to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOV expression or activity. NOV1 is a member of the interferon family. Interferons are part of the body's natural defense to viruses and tumors. Accordingly, diseases associated with aberrant NOV1 expression include viral infections, cancer, neurologic diseases, and acute lymphoblastic leukemia and gliomas. NOV2 is a member of the transmembrane protein family. Aberrant membrane proteins have been identified in a significant number of diseases, including cancer, neurodegenerative disorders, Alzheimer's Disease, Parkinson's Disorder, immune disorders, and hematopoietic disorders. These methods of treatment will be discussed more fully, below.

Disease and Disorders

Diseases and disorders that are characterized by increased (relative to a subject not suffering from the disease or disorder) levels or biological activity can be treated with Therapeutics that antagonize (i.e., reduce or inhibit) activity. Therapeutics that antagonize activity can be administered in a therapeutic or prophylactic manner. Therapeutics that can be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity can be treated with Therapeutics that increase (i.e., are agonists to) activity. Therapeutics that upregulate activity can be administered in a therapeutic or prophylactic manner. Therapeutics that can be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOV expression or activity, by administering to the subject an agent that modulates NOV expression or at least one NOV activity.

NOV1 is a member of the interferon family. Interferons are part of the body's natural defense to viruses and tumors. Accordingly, diseases associated with aberrant NOV1 expression include viral infections, cancer, neurologic diseases, and acute lymphoblastic leukemia and gliomas. NOV2 is a member of the transmembrane protein family. Aberrant membrane proteins have been identified in a significant number of diseases, including cancer, neurodegenerative disorders, Alzheimer's Disease, Parkinson's Disorder, immune disorders, and hematopoietic disorders.

Subjects at risk for a disease that is caused or contributed to by aberrant NOV expression or activity can be identified by, for example, any or a combination of diagnostic or prognostic assays as described herein. Administration of a prophylactic agent can occur prior to the manifestation of symptoms characteristic of the NOV aberrancy, such that a disease or disorder is prevented or, alternatively, delayed in its progression. Depending upon the type of NOV aberrancy, for example, a NOV agonist or NOV antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOV expression or activity for therapeutic purposes. The modulatory method of the invention involves contacting a cell with an agent that modulates one or more of the activities of NOV protein activity associated with the cell. An agent that modulates NOV protein activity can be an agent as described herein, such as a nucleic acid or a protein, a naturally-occurring cognate ligand of a NOV protein, a peptide, a NOV peptidomimetic, or other small molecule. In one embodiment, the agent stimulates one or more NOV protein activity. Examples of such stimulatory agents include active NOV protein and a nucleic acid molecule encoding NOV that has been introduced into the cell. In another embodiment, the agent inhibits one or more NOV protein activity. Examples of such inhibitory agents include antisense NOV nucleic acid molecules and anti-NOV antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOV protein or nucleic acid molecule. In one embodiment, the method involves administering an agent (e.g., an agent identified by a screening assay described herein), or combination of agents that modulates (e.g., up-regulates or down-regulates) NOV expression or activity. In another embodiment, the method involves administering a NOV protein or nucleic acid molecule as therapy to compensate for reduced or aberrant NOV expression or activity.

Stimulation of NOV activity is desirable in situations in which NOV is abnormally downregulated and/or in which increased NOV activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays can be performed with representative cells of the type(s) involved in the patient's disorder, to determine if a given Therapeutic exerts the desired effect upon the cell type(s). Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

Prophylactic and Therapeutic Uses of the Compositions of the Invention

The NOV nucleic acids and proteins of the invention are useful in potential prophylactic and therapeutic applications implicated in a variety of disorders including, but not limited to: those involving development, differentiation, and activation of thymic immune cells; in pathologies related to spermatogenesis and male infertility; diagnosis of several human neoplasias; in diseases or pathologies of cells in blood circulation such as red blood cells and platelets; various immunological disorders and/or pathologies; autoimmune and inflammatory diseases; cardiovascular diseases; metabolic diseases; cancer growth and metastasis; viral infections, cancer therapy, acute lymphoblastic leukemia; gliomas; neurologic diseases; neurodegenerative disorders; Alzheimer's Disease; Parkinson's Disorder; and hematopoietic disorders.

As an example, a cDNA encoding the NOV protein of the invention can be useful in gene therapy, and the protein can be useful when administered to a subject in need thereof. By way of non-limiting example, the compositions of the invention will have efficacy for treatment of patients suffering from the above mentioned disorders.

Both the novel nucleic acid encoding the NOV protein, and the NOV protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies which immunospecifically-bind to the novel substances of the invention for use in therapeutic or diagnostic methods.

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated polypeptide comprising an amino acid sequence selected from the group consisting of:
(a) a mature form of an amino acid sequence selected from the group consisting of SEQ ID NO:2, 4, and 6;
(b) a variant of a mature form of an amino acid sequence selected from the group consisting of SEQ ID NO: 2, 4, and 6, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NO: 2, 4, and 6; and
(d) a variant of an amino acid sequence selected from the group consisting of SEQ ID NO: 2, 4, and 6, wherein one or more amino acid residues in said variant differs from the amino acid sequence of said mature form, provided that said variant differs in no more than 15% of amino acid residues from said amino acid sequence.

2 The polypeptide of claim 1, wherein said polypeptide comprises the amino acid sequence of a naturally-occurring allelic variant of an amino acid sequence selected from the group consisting of SEQ ID NO: 2, 4, and 6.

3. The polypeptide of claim 2, wherein said allelic variant comprises an amino acid sequence that is the translation of a nucleic acid sequence differing by a single nucleotide from a nucleic acid sequence selected from the group consisting of SEQ ID NO:1, 3, and 5.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NO: 2, 4, and 6;
(b) a variant of a mature form of an amino acid sequence selected from the group consisting of SEQ ID NO: 2, 4, and 6, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NO: 2, 4, and 6;
(d) a variant of an amino acid sequence selected from the group consisting of SEQ ID NO: 2, 4, and 6, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NO: 2, 4, and 6, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NO:1, 3,and5.

9. The nucleic acid molecule of claim 5, wherein said nucleic acid molecule comprises a nucleotide sequence selected from the group consisting of
(a) a nucleotide sequence selected from the group consisting of SEQ ID NO: 1, 3, and 5;
(b) a nucleotide sequence differing by one or more nucleotides from a nucleotide sequence selected from the group consisting of SEQ ID NO: 1, 3, and 5, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NO: 1, 3, and 5, or a complement of said nucleotide sequence.

11. The nucleic acid molecule of claim 5, wherein the nucleic acid molecule comprises a nucleotide sequence selected from the group consisting of
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that immunospecifically-binds to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. A method of identifying an agent that binds to a polypeptide of claim 1, the method comprising:
(a) contacting said polypeptide with said agent; and
(b) determining whether said agent binds to said polypeptide.

21. A method for identifying an agent that modulates the expression or activity of the polypeptide of claim 1, the method comprising:
(a) providing a cell expressing said polypeptide;
(b) contacting the cell with said agent; and
(c) determining whether the agent modulates expression or activity of said polypeptide,
whereby an alteration in expression or activity of said peptide indicates said agent modulates expression or activity of said polypeptide.

22. A method for modulating the activity of the polypeptide of claim 1, the method comprising contacting a cell sample expressing the polypeptide of said claim with a compound that binds to said polypeptide in an amount sufficient to modulate the activity of the polypeptide.

23. A method of treating or preventing a NOV-associated disorder, said method comprising administering to a subject in which such treatment or prevention is desired the polypeptide of claim 1 in an amount sufficient to treat or prevent said NOV-associated disorder in said subject.

24. The method of claim 23, wherein said subject is a human.

25. A method of treating or preventing a NOV-associated disorder, said method comprising administering to a subject in which such treatment or prevention is desired the nucleic acid of claim 5 in an amount sufficient to treat or prevent said NOV-associated disorder in said subject.

26. The method of claim 25, wherein said subject is a human.

27. A method of treating or preventing a NOV-associated disorder, said method comprising administering to a subject in which such treatment or prevention is desired the antibody of claim 15 in an amount sufficient to treat or prevent said NOV-associated disorder in said subject.

28. The method of claim 15, wherein the subject is a human.

29. A pharmaceutical composition comprising the polypeptide of claim 1 and a pharmaceutically-acceptable carrier.

30. A pharmaceutical composition comprising the nucleic acid molecule of claim 5 and a pharmaceutically-acceptable carrier.

31. A pharmaceutical composition comprising the antibody of claim 15 and a pharmaceutically-acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. The use of a therapeutic in the manufacture of a medicament for treating a syndrome associated with a human disease, the disease selected from a NOV-associated disorder, wherein said therapeutic is selected from the group consisting of a NOV polypeptide, a NOV nucleic acid, and a NOV antibody.

36. A method for screening for a modulator of activity or of latency or predisposition to a NOV-associated disorder, said method comprising:
(a) administering a test compound to a test animal at increased risk for a NOV-associated disorder, wherein said test animal recombinantly expresses the polypeptide of claim 1;
(b) measuring the activity of said polypeptide in said test animal after administering the compound of step (a);
(c) comparing the activity of said protein in said test animal with the activity of said polypeptide in a control animal not administered said polypeptide, wherein a change in the activity of said polypeptide in said test animal relative to said control animal indicates the test compound is a modulator of latency of or predisposition to a NOV-associated disorder.

37. The method of claim 36, wherein said test animal is a recombinant test animal that expresses a test protein transgene or expresses said transgene under the control of a promoter at an increased level relative to a wild-type test animal, and wherein said promoter is not the native gene promoter of said transgene.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

39. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

40. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NO: 2, 4, and 6, or a biologically active fragment thereof.

41. A method of treating a pathological state in a mammal, the method comprising administering to the mammal the antibody of claim 15 in an amount sufficient to alleviate the pathological state.
